SEQUENCE LISTING (24 sequences)

SEQ ID NO: 1 - protein, 399 aa, Chlamydia pneumoniae
MAASGGTGGLGGTQGVNLAAVEAAAAKADAAEVVASQEGSEMNMIQQSQDLTNPAAATRTKKKEEKFQTLESRKKGEAGKAEKKSESTEEKPDTDLADKYASGNSEISGQELRGLRDAIGDDASPEDILALVQEKIKDPALQSTALDYLVQTTPPSQGKLKEALIQARNTHTEQFGRTAIGAKNILFASQEYADQLNVSPSGLRSLYLEVTGDTHTCDQLLSMLQDRYTYQDMAIVSSFLMKGMATELKRQGPYVPSAQLQVLMTETRNLQAVLTSYDYFESRVPILLDSLKAEGIQTPSDLNFVKVAESYHKIINDKFPTASKVEREVRNLIGDDVDSVTGVLNLFFSALRQTSSRLFSSADKRQQLGAMIANALDAVNINNEDYPKASDFPKPYPWS

SEQ ID NO: 2 - protein, 515 aa, Chlamydia pneumoniae
MTKTEEKPFGKLRSFLWPIHTHELKKVLPMFLMFFCITFNYTVLRDTKDTLIVGAPGSGAEAIPFIKFWLVVPCAIIFMLIYAKLSNILSKQALFYAVGTPFLIFFALFPTVIYPLRDVLHPTEFADRLQAILPPGLLGLVAILRNWTFAAFYVLAELWGSVMLSLMFWGFANEITKIHEAKRFYALFGIGANISLLASGRAIVWASKLRASVSEGVDPWGISLRLLMAMTIVSGLVLMASYWWINKNVLTDPRFYNPEEMQKGKKGAKPKMNMKDSFLYLARSPYILLLALLVIAYGICINLIEVTWKSQLKLQYPNMNDYSEFMGNFSFWTGVVSVLIMLFVGGNVIRKFGWLTGALVTPVMVLLTGIVFFALVIFRNQASGLVAMFGTTPLMLAVVVGAIQNILSKSTKYALFDSTKEMAYIPLDQEQKVKGKAAIDVVAARFGKSGGALIQQGLLVICGSIGAMTPYLAVILLFIIAIWLVSATKLNKLFLAQSALKEQEVAQEDSAPASS

SEQ ID NO: 3 - protein, 619 aa, Chlamydia pneumoniae
MKKGKLGAIVFGLLFTSSVAGFSKDLTKDNAYQDLNVIEHLISLKYAPLPWKELLFGWDLSQQTQQARLQLVLEEKPTTNYCQKVLSNYVRSLNDYHAGITFYRTESAYIPYVLKLSEDGHVFVVDVQTSQGDIYLGDEILEVDGMGIREAIESLRFGRGSATDYSAAVRSLTSRSAAFGDAVPSGIAMLKLRRPSGLIRSTPVRWRYTPEHIGDFSLVAPLIPEHKPQLPTQSCVLFRSGVNSQSSSSSLFSSYMVPYFWEELRVQNKQRFDSNHHIGSRNGFLPTFGPILWEQDKGPYRSYIFKAKDSQGNPHRIGFLRISSYVWTDLEGLEEDHKDSPWELFGEIIDHLEKETDALIIDQTHNPGGSVFYLYSLLSMLTDHPLDTPKHRMIFTQDEVSSALHWQDLLEDVFTDEQAVAVLGETMEGYCMDMHAVASLQNFSQSVLSSWVSGDINLSKPMPLLGFAQVRPHPKHQYTKPLFMLIDEDDFSCGDLAPAILKDNGRATLIGKPTAGAGGFVFQVTFPNRSGIKGLSLTGSLAVRKDGEFIENLGVAPHIDLGFTSRDLQTSRFTDYVEAVKTIVLTSLSENAKKSEEQTSPQETPEVIRVSYPTTTSAS

SEQ ID NO: 4 - protein, 973 aa, Chlamydia pneumoniae
MKTSIRKFLISTTLAPCFASTAFTVEVIMPSENFDGSSGKIFPYTTLSDPRGTLCIFSGDLYIANLDNAISRTSSSCFSNRAGALQILGKGGVFSFLNIRSSADGAAISSVITQNPELCPLSFSGFSQMIFDNCESLTSDTSASNVIPHASAIYATTPMLFTNNDSILFQYNRSAGFGAAIRGTSITIENTKKSLLFNGNGSISNGGALTGSAAINLINNSAPVIFSTNATGIYGGAIYLTGGSMLTSGNLSGVLFVNNSSRSGGAIYANGNVTFSNNSDLTFQNNTASPQNSLPAPTPPPTPPAVTPLLGYGGAIFCTPPATPPPTGVSLTISGENSVTFLENIASEQGGALYGKKISIDSNKSTIFLGNTAGKGGAIAIPESGELSLSANQGDILFNKNLSITSGTPTRNSIHFGKDAKFATLGATQGYTLYFYDPITSDDLSAASAAATVVVNPKASADGAYSGTIVFSGETLTATEAATPANATSTLNQKLELEGGTLALRNGATLNVHNFTQDEKSVVIMDAGTTLATTNGANNTDGAITLNKLVINLDSLDGTKAAVVNVQSTNGALTISGTLGLVKNSQDCCDNHGMFNKDLQQVPILELKATSNTVTTTDFSLGTNGYQQSPYGYQGTWEFTIDTTTHTVTGNWKKTGYLPHPERLAPLIPNSLWANVIDLRAVSQASAADGEDVPGKQLSITGITNFFHANHTGDARSYRHMGGGYLINTYTRITPDAALSLGFGQLFTKSKDYLVGHGHSNVYFATVYSNITKSLFGSSRFFSGGTSRVTYSRSNEKVKTSYTKLPKGRCSWSNNCWLGELEGNLPITLSSRILNLKQIIPFVKAEVAYATHGGIQENTPEGRIFGHGHLLNVAVPVGVRFGKNSHNRPDFYTIIVAYAPDVYRHNPDCDTTLPINGATWTSIGNNLTRSTLLVQASSHTSVNDVLEIFGHCGCDIRRTSRQYTLDIGSKLRF

SEQ ID NO: 5 - protein, 389 aa, Chlamydia pneumoniae
MKKLLKSALLSAAFAGSVGSLQALPVGNPSDPSLLIDGTIWEGAAGDPCDPCATWCDAISLRAGFYGDYVFDRILKVDAPKTFSMGAKPTGSAAANYTTAVDRPNPAYNKHLHDAEWFTNAGFIALNIWDRFDVFCTLGASNGYIRGNSTAFNLVGLFGVKGTTVNANELPNVSLSNGVVELYTDTSFSWSVGARGALWECGCATLGAEFQYAQSKPKVEELNVICNVSQFSVNKPKGYKGVAFPLPTDAGVATATGTKSATINYHEWQVGASLSYRLNSLVPYIGVQWSRATFDADNIRIAQPKLPTAVLNLTAWNPSLLGNATALSTTDSFSDFMQIVSCQINKFKSRKACGVTVGATLVDADKWSLTAEARLINERAAHVSGQFRF

SEQ ID NO: 6 - protein, 165 aa, Chlamydia pneumoniae
MLYWFLSPIMGEDLMAQKEIVSNRKALRNYEVIETLEAGIVLTGTEIKSLRDHGGNLGDAYVIVSKGEGWLLNASIAPYRFGNIYNHEERRKRKLLLHRYELRKLEGKIAQKGMTLIPLGMLLSRGYVKVRLGCCRGKKAYDKRRTIIEREKEREVAAAMKRRHH

SEQ ID NO: 7 - protein, 22 aa, Artificial
  5′ PCR oligonucleotide
GTGCATATGACAAAAACCGAAG

SEQ ID NO: 8 - protein, 25 aa, Artificial
  3′ PCR oligonucleotide
GCTGGATCCACTAAAAAGCAGGTAG

SEQ ID NO: 9 - protein, 20 aa, Artificial
  5′ PCR oligonucleotide
GGAGGCATATGGCAGCATCA

SEQ ID NO: 10 - protein, 28 aa, Artificial
  3′ PCR oligonucleotide
CACAGGATCCGTATTGGTTTTGCATGGC

SEQ ID NO: 11 - protein, 231 aa, Chlamydia pneumoniae
MSKPSPRNANQPQKPSASFNKKTRSRLAELAAQKKAKADDLEQVHPVPTEEEIKKALGNIFEGLSNGLDLQQILGLSDYLLEEIYTVAYTFYSQGKYNEAVGLFQLLAAAQPQNYKYMLGLSSCYHQLHLYNEAAFGFFLAFDAQPDNPIPPYYIADSLLKLQQPEESNNFLDVTMDICGNNPEFKILKERCQIMKQSIEKQMAGETKKAPTKKPAGKSKTTTNKKSGKKR

SEQ ID NO: 12 - protein, 172 aa, Chlamydia pneumoniae
MSHLNYLLEKIAASSKEDFPFPDDLESYLEGYVPDKNIALDTYQKIFKISSEDLEKVYKEGYHAYLDKDYAKSITVFRWLVFFNPFVSKFWFSLGASLHMSEQYSQALHAYGVTAVLRDKDPYPHYYAYICYTLTNEHEEAEKALEMAWVRAQHKPLYNELKEEILDIRKHK

SEQ ID NO: 13 - protein, 556 aa, Chlamydia pneumoniae
MSKLIRRVVTVLALTSMASCFASGGIEAAVAESLITKIVASAETKPAPVPMTAKKVRLVRRNKQPVEQKSRGAFCDKEFYPCEEGRCQPVEAQQESCYGRLYSVKVNDDCNVEICQSVPEYATVGSPYPIEILAIGKKDCVDVVITQQLPCEAEFVSSDPETTPTSDGKLVWKIDRLGAGDKCKITVWVKPLKEGCCFTAATVCACPELRSYTKCGQPAICIKQEGPDCACLRCPVCYKIEVVNTGSAIARNVTVDNPVPDGYSHASGQRVLSFNLGDMRPGDKKVFTVEFCPQRRGQITNVATVTYCGGHKCSANVTTVVNEPCVQVNISGADWSYVCKPVEYSISVSNPGDLVLHDVVIQDTLPSGVTVLEAPGGEICCNKVVWRIKEMCPGETLQFKLVVKAQVPGRFINQVAVTSESNCGTCTSCAETTTHWKGLAATHMCVLDTNDPICVGENTVYRICVTNRGSAEDTNVSLILKFSKELQPIASSGPTKGTISGNTVVFDALPKLGSKESVEFSVTLKGIAPGDARGEAILSSDTLTSPVSDTENTHVY

SEQ ID NO: 14 - protein, 519 aa, Chlamydia pneumoniae
MSEQEKLSNYNADKKLFSGIDKLFQIVKGSYGPKQSLSPTSFFKERGFYAISQTELSNSYENLGVDFAKAMVNKIHKEHSDGATTGLILLHAILQESYAALEKGISTHKLIASLKLQGEKLQEALQQQSWPIKDALKVRNIIFSSLHMPTIADHFYNAFSVVGPEGLISITKERENDKTSMDVFQGFKIPAGYASTYFVSDTASRLTRIAHPLILITDRKISMIHSLLPLLQEISEQNQHLIIFCEDIDPDVLATLVVNKLQGLLQVTVVTIPQLSTTNQELAEDIALFTGTHICPCQEASHVLAPEMVTLGSCLSIEISESQTTLIGGLHIPEVLTLKTRQLAEEIRTTSCLETKKRLIKSTNRLQSSVAILPTDEDNEPLYTLALKIMESALSRGYVPGGGVALFYASLTLGTPKDDADENSIAISLLQKACCAPLKLLATNADLDGDAVIAKLSSLGTTSLGISVFSREIEDLIAGGILDSLATTSTILAQALDTAILVLSSKILILENQYEISTL

SEQ ID NO: 15 - DNA, 1200 bp, Chlamydia pneumoniae
atggcagcatcaggaggcacaggtggtttaggaggcactcagggtgtcaaccttgcagctgtagaagctgcagctgcaaaagcagatgcagcagaagttgtagccagccaagaaggttctgagatgaacatgattcaacaatctcaggacctgacaaatcccgcagcagcaacacgcacgaaaaaaaaggaagagaagtttcaaactctagaatctcggaaaaaaggagaagctggaaaggctgagaaaaaatctgaatctacagaagagaagcctgacacagatcttgctgataagtatgcttctgggaattctgaaatctctggtcaagaacttcgcggcctgcgtgatgcaataggagacgatgcttctccagaagacattcttgctcttgtacaagagaaaattaaagacccagctctgcaatccacagctttggactacctggttcaaacgactccaccctcccaaggtaaattaaaagaagcgcttatccaagcaaggaatactcatacggagcaattcggacgaactgctattggtgcgaaaaacatcttatttgcctctcaagaatatgcagaccaactgaatgtttctccttcagggcttcgctctttgtacttagaagtgactggagacacacatacctgtgatcagctactttctatgcttcaagaccgctatacctaccaagatatggctattgtcagctcctttctaatgaaaggaatggcaacagaattaaaaaggcagggtccctacgtacccagtgcgcaactacaagttctcatgacagaaactcgtaacctgcaagcagttcttacctcgtacgattactttgaaagtcgcgttcctattttactcgatagcttaaaagctgagggaatccaaactccttctgatctaaactttgtgaaggtagctgagtcctaccataaaatcattaacgataagttcccaacagcatctaaagtagaacgagaagtccgcaatctcataggagacgatgttgattctgtgaccggtgtcttgaacttattcttttctgctttacgtcaaacgtcgtcacgccttttctcttcagcagacaaacgtcagcaattaggagctatgattgctaatgctttagatgctgtaaatataaacaatgaagattatcccaaagcatcagacttccctaaaccctatccttggtcatga

SEQ ID NO: 16 - DNA, 1548 bp, Chlamydia pneumoniae
atgacaaaaaccgaagaaaaaccttttggaaaattgcgctctttcttgtggccgatacatactcacgagctaaagaaagttctgccaatgttcctaatgttcttctgtattacatttaactatacggtgttacgcgatacaaaagacactcttattgtgggagctcctggttctggtgcagaggcaatacctttcatcaagttttggcttgttgtcccctgtgctattatctttatgcttatttatgcaaagctaagtaatattttaagtaagcaggccttattttatgcagtgggaacgccctttttaattttctttgccctgttcccgactgtaatttatccgctacgcgatgttttacatcctacagaatttgctgaccgtttacaggccatcctacctccaggattgctaggactcgttgccatcttaagaaactggacatttgctgcattttatgtacttgctgaactatggggaagcgtcatgctatctctaatgttctggggatttgctaatgaaattacaaaaatccacgaagcaaagcgtttctacgctcttttcggtatcggagctaatatttctttactagcttctggtcgtgcaattgtttgggcttcaaagttgagagcttccgtttctgaaggtgtagatccttggggaatttctttacgtcttttgatggctatgactattgtatctggacttgttcttatggccagttactggtggatcaataagaacgtattgaccgatcctcgcttctataatccagaagaaatgcaaaaggggaaaaaaggtgctaaacctaaaatgaatatgaaagatagcttcctctatcttgctagatctccttatattcttttattagctctcttggttattgcctatggtatttgcattaacttaatcgaagtgacttggaaaagtcagctgaaactgcaatatcctaatatgaatgactatagtgagttcatggggaacttctccttctggactggcgtagtatccgtacttatcatgctatttgttggtggtaacgtcattcgtaaatttggatggttaactggagccctagtcactcctgtcatggttctcctaacaggtatcgttttcttcgctcttgttatctttagaaaccaagcttctgggctggtcgctatgttcggtacaactcctctcatgctagctgtggttgtcggagctatacagaatattctttcgaaatccacaaaatacgctctctttgactcaactaaagaaatggcctatatccctcttgaccaagagcaaaaagtcaaaggtaaggctgctattgatgtagttgctgcccgcttcggaaaatcaggaggagctttaatccaacaaggtttgctcgttatctgtggaagtattggagctatgaccccttatcttgcagtgattcttcttttcatcattgctatttggttggtttctgcaactaagttaaacaaactattcttagcgcagtctgctcttaaagaacaagaagtggctcaagaagattcagctcctgcttcttcatag

SEQ ID NO: 17 - DNA, 1860 bp, Chlamydia pneumoniae
atgaaaaaagggaaattaggagccatagtttttggccttctatttacaagtagtgttgctggtttttctaaggatttgactaaagacaacgcttatcaagatttaaatgtcatagagcatttaatatcgttaaaatatgctcctttaccatggaaggaactattatttggttgggatttatctcagcaaacacagcaagctcgcttgcaactggtcttagaagaaaaaccaacaaccaactactgccagaaggtactctctaactacgtgagatcattaaacgattatcatgcagggattacgttttatcgtactgaaagtgcgtatatcccttacgtattgaagttaagtgaagatggtcatgtctttgtagtcgacgtacagactagccaaggggatatttacttaggggatgaaatccttgaagtagatggaatggggattcgtgaggctatcgaaagccttcgctttggacgagggagtgccacagactattctgctgcagttcgttccttgacatcgcgttccgccgcttttggagatgcggttccttcaggaattgccatgttgaaacttcgccgacccagtggtttgatccgttcgacaccggtccgttggcgttatactccagagcatatcggagatttttctttagttgctcctttgattcctgaacataaacctcaattacctacacaaagttgtgtgctattccgttccggggtaaattcacagtcttctagtagctctttattcagttcctacatggtgccttatttctgggaagaattgcgggttcaaaataagcagcgttttgacagtaatcaccatatagggagccgtaatggatttttacctacgtttggtcctattctttgggaacaagacaaggggccctatcgttcctatatctttaaagcaaaagattctcagggcaatccccatcgcataggatttttaagaatttcttcttatgtttggactgatttagaaggacttgaagaggatcataaggatagtccttgggagctctttggagagatcatcgatcatttggaaaaagagactgatgctttgattattgatcagacccataatcctggaggcagtgttttctatctctattcgttactatctatgttaacagatcatcctttagatactcctaaacatagaatgattttcactcaggatgaagtcagctcggctttgcactggcaagatctactagaagatgtcttcacagatgagcaggcagttgccgtgctaggggaaactatggaaggatattgcatggatatgcatgctgtagcctctcttcaaaacttctctcagagtgtcctttcttcctgggtttcaggtgatattaacctttcaaaacctatgcctttgctaggatttgcacaggttcgacctcatcctaaacatcaatatactaaacctttgtttatgttgatagacgaggatgacttctcttgtggagatttagcgcctgcaattttgaaggataatggccgcgctactctcattggaaagccaacagcaggagctggaggttttgtattccaagtcactttccctaaccgttctggaattaaaggtctttctttaacaggatctttagctgttaggaaagatggtgagtttattgaaaacttaggagtggctcctcatattgatttaggatttacctccagggatttgcaaacttccaggtttactgattacgttgaggcagtgaaaactatagttttaacttctttgtctgagaacgctaagaagagtgaagagcagacttctccgcaagagacgcctgaagttattcgagtctcttatcccacaacgacttctgcttcgtaa

SEQ ID NO: 18 - DNA, 2922 bp, Chlamydia pneumoniae
atgaaaacgtctattcgtaagttcttaatttctaccacactggcgccatgttttgcttcaacagcgtttactgtagaagttatcatgccttccgagaactttgatggatcgagtgggaagatttttccttacacaacactttctgatcctagagggacactctgtattttttcaggggatctctacattgcgaatcttgataatgccatatccagaacctcttccagttgctttagcaatagggcgggagcactacaaatcttaggaaaaggtggggttttctccttcttaaatatccgttcttcagctgacggagccgcgattagtagtgtaatcacccaaaatcctgaactatgtcccttgagtttttcaggatttagtcagatgatcttcgataactgtgaatctttgacttcagatacctcagcgagtaatgtcatacctcacgcatcggcgatttacgctacaacgcccatgctctttacaaacaatgactccatactattccaatacaaccgttctgcaggatttggagctgccattcgaggcacaagcatcacaatagaaaatacgaaaaagagccttctctttaatggtaatggatccatctctaatggaggggccctcacgggatctgcagcgatcaacctcatcaacaatagcgctcctgtgattttctcaacgaatgctacagggatctatggtggggctatttaccttaccggaggatctatgctcacctctgggaacctctcaggagtcttgttcgttaataatagctcgcgctcaggaggcgctatctatgctaacggaaatgtcacattttctaataacagcgacctgactttccaaaacaatacagcatctccacaaaactccttacctgcacctacacctccacctacaccaccagcagtcactcctttgttaggatatggaggcgccatcttctgtactcctccagctacccccccaccaacaggtgttagcctgactatatctggagaaaacagcgttacattcctagaaaacattgcctccgaacaaggaggagccctctatggcaaaaagatctctatagattctaataaatctacaatatttcttggaaatacagctggaaaaggaggcgctattgctattcccgaatctggggagctctctctatccgcaaatcaaggtgatatcctctttaacaagaacctcagcatcactagtgggacacctactcgcaatagtattcacttcggaaaagatgccaagtttgccactctaggagctacgcaaggctataccctatacttctatgatccgattacatctgatgatttatctgctgcatccgcagccgctactgtggtcgtcaatcccaaagccagtgcagatggtgcgtattcagggactattgtcttttcaggagaaaccctcactgctaccgaagcagcaacccctgcaaatgctacatctacattaaaccaaaagctagaacttgaaggcggtactctcgctttaagaaacggtgctaccttaaatgttcataacttcacgcaagatgaaaagtccgtcgtcatcatggatgcagggaccacattagcaactacaaatggagctaataatactgacggtgctatcaccttaaacaagcttgtaatcaatctggattctttggatggcactaaagcggctgtcgttaatgtgcagagtaccaatggagctctcactatatccggaactttaggacttgtgaaaaactctcaagattgctgtgacaaccacgggatgtttaataaagatttacagcaagttccgattttagaactcaaagcgacttcaaatactgtaaccactacggacttcagtctcggcacaaacggctatcagcaatctccctatgggtatcaaggaacttgggagtttaccatagacacgacaacccatacggtcacaggaaattggaaaaaaaccggttatcttcctcatccggagcgtcttgctcccctcattcctaatagcctatgggcaaacgtcatagatttacgagctgtaagtcaagcgtcagcagctgatggcgaagatgtccctgggaagcaactgagcatcacaggaattacaaatttcttccatgcgaatcataccggtgatgcacgcagctaccgccatatgggtggaggctacctcatcaatacctacacacgcatcactccagatgctgcgttaagtctaggttttggacagctgtttacaaaatctaaggattacctcgtaggtcacggtcattctaacgtttatttcgctacagtatactctaacatcaccaagtctctgtttggatcatcgagattcttctcaggaggcacttctcgagttacctatagccgtagcaatgagaaagtaaagacttcatatacaaaattgcctaaagggcgctgctcttggagtaacaattgctggttaggagaactcgaagggaaccttcccatcactctctcttctcgcatcttaaacctcaagcagatcattccctttgtaaaagctgaagttgcttacgcgactcatgggggcatccaagaaaatacccctgaggggaggatttttggacacggtcatctactcaacgttgcagttcccgtaggcgtccgctttggtaaaaattctcataatcgaccagatttttacactataatcgtagcctatgctcctgatgtctatcgtcacaatcctgattgcgatacgacattacctattaatggagctacgtggacctctatagggaataatctaaccagaagtactttgctagtacaagcatccagccatacttcagtaaatgatgttctagagatcttcgggcactgtggatgtgatattcgcagaacctcccgtcaatatactctagatataggaagcaaattacgattttaa

SEQ ID NO: 19 - DNA, 1170 bp, Chlamydia pneumoniae
atgaaaaaactcttaaagtcggcgttattatccgccgcatttgctggttctgtcggctccttacaagccttgcctgtagggaacccttctgatccaagcttattaattgatggtacaatatgggaaggtgctgcaggagatccttgcgatccttgcgctacttggtgcgacgctattagcttacgtgctggattttacggagactatgttttcgaccgtatcttaaaagtagatgcacctaaaacattttctatgggagccaagcctactggatccgctgctgcaaactatactactgccgtagatagacctaacccggcctacaataagcatttacacgatgcagagtggttcactaatgcaggcttcattgccttaaacatttgggatcgctttgatgttttctgtactttaggagcttctaatggttacattagaggaaactctacagcgttcaatctcgttggtttattcggagttaaaggtactactgtaaatgcaaatgaactaccaaacgtttctttaagtaacggagttgttgaactttacacagacacctctttctcttggagcgtaggcgctcgtggagccttatgggaatgcggttgtgcaactttgggagctgaattccaatatgcacagtccaaacctaaagttgaagaacttaatgtgatctgtaacgtatcgcaattctctgtaaacaaacccaagggctataaaggcgttgctttccccttgccaacagacgctggcgtagcaacagctactggaacaaagtctgcgaccatcaattatcatgaatggcaagtaggagcctctctatcttacagactaaactctttagtgccatacattggagtacaatggtctcgagcaacttttgatgctgataacatccgcattgctcagccaaaactacctacagctgttttaaacttaactgcatggaacccttctttactaggaaatgccacagcattgtctactactgattcgttctcagacttcatgcaaattgtttcctgtcagatcaacaagtttaaatctagaaaagcttgtggagttactgtaggagctactttagttgatgctgataaatggtcacttactgcagaagctcgtttaattaacgagagagctgctcacgtatctggtcagttcagattctaa

SEQ ID NO: 20 - DNA, 498 bp, Chlamydia pneumoniae
atgttatactggtttttgtccccaattatgggggaggatcttatggcacaaaaagaaattgtttctaatcgcaaggctctgcgtaactatgaagttatagagactttagaagcaggcatcgttttgactgggactgagattaagtcgttgcgcgatcatgggggaaacctcggtgatgcttatgtcattgtttctaaaggtgaggggtggttattaaacgcgagtattgctccctatcggtttggaaatatctataaccatgaggagcgtcgtaaacgtaaactccttcttcatagatatgaacttcgtaagttagagggtaagattgctcaaaagggcatgactttgattcctctgggaatgttgctgagtcgcggctatgttaaggtacgtttgggttgttgtcgtgggaaaaaagcttatgataagcgtcgtacgatcatagaaagagaaaaggaacgtgaagttgccgctgctatgaagaggcgccatcattga

SEQ ID NO: 21 - DNA, 696 bp, Chlamydia pneumoniae
atgagcaagccctctcctcgtaatgccaatcaacctcaaaaaccttcagcctctttcaataaaaaaacgcgaagccgtctagcagagctggctgctcaaaagaaagcaaaggctgatgacttagaacaagtccatcccgtacctacggaagaagaaattaaaaaagctttaggcaatatcttcgaaggtcttagcaatggactagacctacaacagattctaggtctctcggactatctcttagaggagatctatactgtagcttatacattctattctcaagggaagtacaacgaagctgtaggactcttccagttattggcagcagcacaacctcagaactacaagtatatgttagggttaagctcctgctaccaccaattgcatttatataatgaagcggcttttggatttttccttgctttcgatgctcaacctgacaacccaattcctccttactacattgctgatagcttattgaagctacagcaacccgaagaatctaacaatttcttagacgtcaccatggatatctgtgggaataacccagaattcaaaatcttaaaagaacgttgccaaattatgaaacagtctattgagaagcagatggctggagaaactaagaaagcaccgacaaaaaaacctgcgggaaaaagcaaaacaactacaaataagaaaagcggaaagaaacgttag

SEQ ID NO: 22 - DNA, 519 bp, Chlamydia pneumoniae
atgtcacatttaaattatttactagaaaaaatcgctgcatcttccaaggaagacttccctttcccagatgatttggaaagctatttggaaggatatgtcccggataaaaatatagctttagatacctaccaaaagatcttcaagatatcctccgaggatcttgaaaaagtatataaagagggttatcacgcctatctcgacaaagactatgctaaaagcatcaccgtttttcgctggttagttttcttcaatccttttgtctctaagttttggttttctttaggtgcctctctacatatgtccgaacaatattcacaagctttgcatgcctatggagtcactgcagtccttcgagataaagatccctatcctcattactatgcctacatatgctatactcttacaaatgaacatgaagaagctgaaaaagctttagaaatggcgtgggtacgtgcacaacacaagcccctctataatgaattgaaagaagaaattctagatattcgaaaacataaataa

SEQ ID NO: 23 - DNA, 1671 bp, Chlamydia pneumoniae
atgtccaaactcatcagacgagtagttacggtccttgcgctaacgagtatggcgagttgctttgccagcgggggtatagaggccgctgtagcagagtctctgattactaagatcgtcgctagtgcggaaacaaagccagcacctgttcctatgacagcgaagaaggttagacttgtccgtagaaataaacaaccagttgaacaaaaaagccgtggtgctttttgtgataaagaattttatccctgtgaagagggacgatgtcaacctgtagaggctcagcaagagtcttgctacggaagattgtattctgtaaaagtaaacgatgattgcaacgtagaaatttgccagtccgttccagaatacgctactgtaggatctccttaccctattgaaatccttgctataggcaaaaaagattgtgttgatgttgtgattacacaacagctaccttgcgaagctgaattcgtaagcagtgatccagaaacaactcctacaagtgatgggaaattagtctggaaaatcgatcgcctgggtgcaggagataaatgcaaaattactgtatgggtaaaacctcttaaagaaggttgctgcttcacagctgctactgtatgtgcttgcccagagctccgttcttatactaaatgcggtcaaccagccatttgtattaagcaagaaggacctgactgtgcttgcctaagatgccctgtatgctacaaaatcgaagtagtgaacacaggatctgctattgcccgtaacgtaactgtagataatcctgttcccgatggctattctcatgcatctggtcaaagagttctctcttttaacttaggagacatgagacctggcgataaaaaggtatttacagttgagttctgccctcaaagaagaggtcaaatcactaacgttgctactgtaacttactgcggtggacacaaatgttctgcaaatgtaactacagttgttaatgagccttgtgtacaagtaaatatctctggtgctgattggtcttacgtatgtaaacctgtggagtactctatctcagtatcgaatcctggagacttggttcttcatgatgtcgtgatccaagatacactcccttctggtgttacagtactcgaagctcctggtggagagatctgctgtaataaagttgtttggcgtattaaagaaatgtgcccaggagaaaccctccagtttaaacttgtagtgaaagctcaagttcctggaagattcataaatcaagttgcagtaactagtgagtctaactgcggaacatgtacatcttgcgcagaaacaacaacacattggaaaggtcttgcagctacccatatgtgcgtattagacacaaatgatcctatctgtgtaggagaaaatactgtctatcgtatctgtgtaactaaccgtggttctgctgaagatactaacgtatctttaatcttgaagttctcaaaagaacttcagccaatagcttcttcaggtccaactaaaggaacgatttcaggtaataccgttgttttcgacgctttacctaaactcggttctaaggaatctgtagagttttctgttaccttgaaaggtattgctcccggagatgctcgcggcgaagctattctttcttctgatacactgacttcaccagtatcagacacagaaaatacccacgtgtattaa

SEQ ID NO: 24 - DNA, 1560 bp, Chlamydia pneumoniae
atgtccgaacaggaaaaattatctaattacaatgctgataaaaaacttttttcagggatagataaacttttccagatagtaaagggatcgtatggtcccaagcaatccctctccccaacttctttctttaaagagcgtggcttctacgctatttcacaaacagaactttcgaattcctatgaaaatctcggtgtagattttgcgaaagctatggtgaataaaattcataaggaacacagcgacggcgcgactactggacttattttactccatgcaattttacaggaaagctatgcagctttagaaaagggaatctccacacacaagctcattgcctctttaaaattgcagggagaaaagcttcaggaagccttacaacaacaatcttggcctattaaagatgctctaaaagttcgcaatattattttttcttctctacacatgcccaccattgccgatcatttttacaatgctttttctgtggtaggccctgagggtcttatctccatcactaaggagagagaaaacgacaaaacttccatggatgtctttcaaggatttaaaattcctgcagggtatgcttcgacgtattttgtttctgatacagcgtctcgtctaactagaattgctcatcctctaattctgatcacagatagaaagatctctatgattcactccttacttcccctactccaagaaatttcagaacaaaaccagcacctcatcattttctgtgaagatatcgatccagacgtacttgctacccttgttgttaacaaattgcaagggctcttgcaagtaactgtagtcaccattcctcaactctctactacaaatcaagaattagccgaggatattgctttatttactgggacccacatttgtccttgccaagaagcatctcatgttttagctcctgagatggtcactttagggtcctgtctatctatagaaatctcagagtcacaaacgacactcattggaggcctacatatcccggaagtcctgaccttaaagacgcgacaattagcagaagaaatacgcacgacctcatgccttgaaacaaaaaaaaggctaatcaaaagcacaaataggctccaaagttcggtagccattcttcctactgatgaagataacgaacctttatataccctcgctcttaagattatggaatctgctttaagtcgtgggtatgttcctggaggaggcgttgcattgttctatgcatctttaactttagggactcctaaagatgatgctgatgagaatagtattgcaatttctctcttacagaaggcctgttgtgctcctctgaagctcttggcaaccaatgcagacttggatggggatgccgtgattgctaagctctcctcacttggaacaacaagcctaggaatcagtgtgttttctagagaaattgaagatcttattgctggtggaatcttagattcgttagctacgacatctacaatcctagcccaagccttagatacagcgattctcgttctctcttctaaaattttaattttagaaaatcaatatgagatttctacgctctga

(19) United States
(12) Patent Application Publication
(10) Pub. No.: US 2009/0252765 A1
(43) Pub. Date: Oct. 8, 2009

(54) BACILLE CALMETTE-GUERIN (BCG)-BASED ANTI-ATHEROMA VACCINE AND METHODS OF USE THEREOF

(75) Inventors: Katalin Burian, Szeged (HU); Valeria Endresz, Szeged (HU); Vijay Kakkar, London (GB); Xinjie Lu, London (GB); Andras Miczak, Szeged (HU)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignees: Thrombosis Research Institute, London (GB); University of Szeged, Dugonics ter (HU)

(21) Appl. No.: 12/122,454

(22) Filed: May 16, 2008

(30) Foreign Application Priority Data
May 16, 2007 (GB) GB 0709373.5

(51) Int. Cl. A61K 39/04; C12N 1/21; C12N 15/63; A61K 39/118

(52) U.S. Cl. 424/248.1; 435/252.3; 435/320.1; 424/263.1

(57) ABSTRACT

The invention relates to a recombinant Mycobacterium bovis which expresses at least one Chlamydophila pneumoniae antigen.

### CROSS-REFERENCE TO RELATED APPLICATION

This application claims priority to GB patent application No. GB 0709373.5, filed on May 16, 2007, which is herein incorporated by reference in its entirety.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 344306SequenceListing.txt, a creation date of May 16, 2008, and a size of 57.9 KB. The sequence listing filed via EFS-Web is part of the specification and is hereby incorporated in its entirety by reference herein.

### FIELD OF THE INVENTION

The present invention relates to a recombinant Mycobacterium bovis which expresses at least one Chlamydophila pneumoniae antigen. More particularly, the invention relates to a recombinant Mycobacterium bovis which expresses an antigenically distinct part of one or more of: CopN, CPAF, ADP/ATP Translocase I, SmpB, pmpD, MOMP, LcrH1, LcrH2, OMP2 and HSP60.

### BACKGROUND OF THE INVENTION

Cardiovascular disease (CVD) is a ubiquitous cause of death and disability, being responsible for almost 50% of all deaths; two and a half times more than all types of cancer (21%) put together. Heart attack (CHD) and stroke are two of its most important manifestations, others being occlusive peripheral vascular disease (PVD) and venous thromboembolism.

Atheroma (e.g. atherosclerosis) is a progressive disease best described according to the classification of the American Heart Association's Committee on Vascular Lesions (Stary et al. Arterioscler Thromb 1994, 14 840-856; Stary et al. Arterioscler Thromb Vasc Biol 1995 15: 1512-1531). At each stage of atherothrombosis, inflammation has been shown to play a role (Pesonen et al, Atherosclerosis. 1999, 142, 425-429). Following injury by ox-LDL or other agents, monocytes bind at the site of the developing lesion. The adherent monocytes move into the vessel wall where they continue to ingest modified lipids and lipoproteins as they differentiate into macrophages and eventually become foam cells—macrophage cells constitute more than half of all the cells that may be released from rupture of a mature plaque. T cells and mast cells also accumulate within the lesion. Vessel wall SMCs begin to migrate and synthesise collagen while producing factors that recruit additional monocytes. Activation of the macrophages, T-lymphocytes and SMCs leads to the release of cytokines, chemokines and growth factors. In particular IL-6 is synthesized stimulating an increase in blood levels of fibrinogen, PAI-1 and C-reactive protein, as well as tissue factor (Paoletti et al, Circulation. 2004, 109 (23 Suppl 1): III 20-6). Other inflammatory cytokines such as IL-1 and TNF induce the expression of cellular adhesion molecules stimulating further adhesion of leukocytes to the endothelium. Metalloproteinases synthesised by resident macrophages digest the fibrous cap of the plaque enhancing the risk of rupture and these enzymes may also be responsible for the cleavage of the adhesion molecules from damaged endothelium-soluble forms of which are increased in the blood of atherosclerotic patients (E-selectin, VCAM and ICAM) (Roldan et al., Thromb Haemost. 2003, 90, 1007-1020). Reduced NO production by the damaged endothelium leads to further platelet adherences and aggregation. The final phase of the inflammatory process occurs when the plaque ruptures releasing a large mass of prothrombotic material into blood with resulting thrombosis.

It is likely that the chronic inflammation occurring in atherosclerosis is maintained by repeated and/or chronic infections (Epstein et al, Arterioscler Thromb Vasc Biol. 2000, 20, 1417-1420) and there seems to be a relationship between infective burden, extent of atherosclerosis and clinical prognosis (Ferns et al, Science. 1991, 253, 1129-1132). Seroreactivity against a number of bacterial and viral organisms is associated with advanced atherosclerosis and chronic infection is significantly associated with increased death (Zhu et al, Circulation. 2001, 103, 45-51).

Chlamydophila (formerly Chlamydia) pneumoniae is a common respiratory pathogen. It is capable of infecting the principal cell types found in lesions and has been found atheromatous tissue obtained from endarterectomy and restenotic bypass (Gaydos et al., Infect Immun. 1996, 64, 1614-1620; Chiu et al, Circulation. 1997, 96, 2144-2148; Maass et al., J Am Coll Cardiol. 1998, 31, 827-832). C. pneumoniae-serospecific IgG has been observed in patients with acute or chronic coronary artery disease (Saikku et al., Lancet 1988 2: 983-986) Chlamydophila pneumoniae is a gram-negative obligate intracellular bacterial pathogen that causes both respiratory and systemic diseases of humans. In particular, Chlamydia pneumoniae is thought to be implicated in approximately 7 to 10% of cases of community-acquired pneumoniae among adults. Chlamydia pneumoniae has also been associated with other respiratory tract diseases such as bronchitis, sinusitis, asthmatic bronchitis, adult-onset asthma, and chronic obstructive pulmonary disease. In addition to acute infections, such as pneumonia or other respiratory diseases (Grayston, et al., 1992, 1993), C. pneumoniae has been associated with a wide range of chronic diseases that are characterized by a local and/or systemic inflammatory response (Muhlestein, 1996; Ramirez, et al., 1996). The chlamydial developmental cycle begins when the extracellular elementary body (EB) attaches and enters the host cell by endocytosis and is contained within an inclusion, which continues to enlarge, while the EB differentiates into the reticulate body (RB). The RB undergoes logarithmic division by binary fission and subsequently redifferentiates into an EB. These infectious EBs are released by host cell lysis at 60 to 84 h postinfection (hpi) and initiate a new cycle of replication (Wolf, et al., 2000). Several in vitro models of chlamydial persistence have been established to mimic chlamydial persistence in vivo. Helicobacter pylori, a resident of human gastric epithelium, is implicated in several serological studies, which indicate a significant association with atherosclerotic disease (Espinola-Klein et al, Circulation. 2002, 105, 15-21; Georges et al., Am J Cardiol. 2003, 92, 515-521; Huittinen et al, Circulation. 2003, 107, 2566-2570).

Human cytomegalovirus (HMCV) is a member of the herpesvirus family, several members of which show a strong association with the cardiovascular system. Several seroepidemiological studies have shown a link to atherosclerosis (Zhou et al, N Engl J Med 1996 335: 624-630; Adam et al Lancet. 1987 2: 291-293). Moreover there is direct evidence of the virus in arteries from patients (Hendrix et al., Am J Pathol 1989 134: 1151-1157). HMCV can infect all types of cell types in the vascular wall, stimulating the synthesis of chemokines and cytokines (Jarvis & Nelson, Opin Microbiol 2002 5:403-407).

It has been suggested that cross-reactivity between antigens from a foreign agent with self-proteins may trigger autoimmune diseases. An immune response against a shared epitope can evoke a tissue-specific immune response that is thought to be capable of eliciting cell and tissue destruction. This is brought about generating cytotoxic cross-reactive effector lymphocytes or antibodies that recognise specific determinants on target cells. By a complementary mechanism, the microbe can induce cellular injury and release self-antigens, which generate immune responses that cross-react with additional but genetically distinct self-antigens. Infection of cells in the vascular wall may initiate a cascade of inflammatory reactions and may lead to the destruction of cells via direct cytopathicity. Atherosclerosis in hypercholesterolaemic low-density lipoprotein receptor (LDLR) mice is significantly reduced in the absence of monocyte chemoattactant protein-1 (MCP-1) (Gu et al, Mol. Cell. 1998, 2, 275-281) or the combined absence of P- and E-selectins (Dong, et al, J. Clin. Invest. 1998, 102, 145-152). Pathogen-specific immune responses are induced when the infectious agent or its antigens reach secondary lymphoid organs (Karrer et al, J. Exp. Med. 1997, 185, 2157-2170). Cross-reactive Th cells recognising microbial and self-antigens may be generated during viral and bacterial infections (Oldstone, Cell 1987, 50, 819-820). The peripheral activation of pathogen-induced Th cells, recognising self-antigens presented by tissue-resident APC, may result in the release of cytokines such as IFN-γ and chemokines, which attract further T cells and macrophages to the vascular lesion. Several C. pneumoniae antigens have been identified that induce protective immunity in a mouse model of acute C. pneumoniae infection (Murdin, J. Infect. Dis. 2000, 181 (Suppl. 3), S544-S551).

In addition a number of other approaches to developing anti-atheroma vaccines based on autoantigens, particularly those related to lipid metabolism, have previously been investigated. T cells extracted from human atherosclerotic plaques have been shown to recognise oxidised low density lipoprotein (oxLDL) and immunisation of LDL-deficient rabbits with molondialdehyde-modified LDL was shown to reduce atherosclerosis (Palinski et al, Proc. Natl. Acad. Sci. USA 1995, 92, 821-825). International application WO 01/68119 discloses antigenic compositions based on such modified LDL or apoB100 immunogens. Another target for such approaches is cholesterol ester transferase protein (CETP) modulation of the activity of which may be beneficial in preventing the progression of atherogenesis. International application WO 97/41227 discloses a DNA vaccine approach using a plasmid encoding various T and B-cell CETP epitopes. Attempts have also been made to target cholesterol directly (WO 92/10203).

Vaccines essentially deliver immunogenic epitopes to the immune system via antigen presenting cells in a way that encourages the development of long-term protective immunity. The immunogen may be in the form of a purified or semi-purified protein or peptide, with or without an adjuvant; as part of a live attenuated, or killed, infectious organism; or may be encoded in a nucleic acid delivery vector (reviewed in Raychaudhuri and Rock, 1998, Nature Biotechnology 16: 1025-1031). The very first vaccines were based on live attenuated organisms and the bacille Calmette-Guérin (BCG) attenuated strain of Mycobacterium bovis has been used as a protective vaccine against tuberculosis since 1921. However, recently, the technology allowing the generation of genetically manipulated BCG expressing a variety of potentially useful immunogens has been developed. (reviewed in Hanson et al 1995, Ann NY Acad Sci 754:214-221). BCG has a number of advantages, in addition to its long clinical history. It has a powerful adjuvant effect and is persistent, providing a long period of immunisation. M bovis is also an intracellular parasite of macrophages and so BCG offers the possibility of delivering immunogens directly to antigen-presenting cells. The development of shuttle vectors allowing conventional construction and growth of expression vectors in E coli, which could then be used to transform BCG, allowed the generation of recombinant BCG (rBCG) vaccines for a variety of potential targets (Jacobs et al 1987, Nature 357: 532; Stover et a/1991 Nature 351: 456). A further aspect of the intrinsic adjuvant effect of BCG relates to the immunostimulatory properties of CpG-rich bacterial DNA. Conserved microbial motifs, known as pathogen-associated molecular patterns (PAMPs) are recognised by a range of pattern-recognition receptors expressed by cells of the immune system, which are distinct from the hypervariable B- and T-cell receptors that from the basis of the adaptive immune response. Unmethylated CpG dinucleotides are common in bacterial DNA and are recognised by the Toll-like receptor 9 (TLR9) (Hemmi et al, 2000, Nature 7:740-5) in a complex and sequence-specific way (Kindrachuk et al, 2007, J Biol Chem 282:13944).

rBCG vaccines may express immunogenic peptides either as secreted proteins or as cell surface chimeric proteins, glycoproteins or lipoproteins. Among the vaccines depending on cell surface expression of immunogens are rBCG expressing the MUC1 mucin tumour antigen (He et al, 2002, Int J Oncology 20: 1305-1311) and the Schistosoma mansoni Sm 14 antigen (Varaldo et al 2004, Infection and Immunity 72: 3336-3343).

Accordingly, there remains a need for an improved vaccine that is capable of providing long-term protective immunity against Chlamydophila pneumoniae infection.

### SUMMARY OF INVENTION

In a first aspect the invention provides a recombinant Mycobacterium bovis which expresses at least one Chlamydophila pneumoniae antigen. Preferably, said antigen comprises at least an antigenically distinct part of one or more of: CopN, CPAF, ADP/ATP Translocase I, SmpB, pmpD, MOMP, LcrH1, LcrH2, OMP2 and HSP60. More referably, said antigen comprises at least an antigenically distinct part of ADP/ATP translocase I.

In one embodiment, the Mycobacterium bovis expresses an antigenically distinct part of a polypeptide selected from the group consisting of:
i) a polypeptide having an amino acid sequence at least 70% identical to SEQ ID NO:1;
ii) a polypeptide having an amino acid sequence at least 70% identical to SEQ ID NO:2;
iii) a polypeptide having an amino acid sequence at least 70% identical to SEQ ID NO:3;
iv) a polypeptide having an amino acid sequence at least 70% identical to SEQ ID NO:4;
v) a polypeptide having an amino acid sequence at least 70% identical to SEQ ID NO:5;
vi) a polypeptide having an amino acid sequence at least 70% identical to SEQ ID NO:6;
vii) a polypeptide having an amino acid sequence at least 70% identical to SEQ ID NO:11;
viii) a polypeptide having an amino acid sequence at least 70% identical to SEQ ID NO:12;
ix) a polypeptide having an amino acid sequence at least 70% identical to SEQ ID NO:13; and
x) a polypeptide having an amino acid sequence at least 70% identical to SEQ ID NO:14.

In a further embodiment, the Mycobacterium bovis expresses an antigenically distinct part of a polypeptide selected from the group consisting of:
i) a polypeptide having an amino acid sequence of SEQ ID NO:1;
ii) a polypeptide having an amino acid sequence of SEQ ID NO:2;
iii) a polypeptide having an amino acid sequence of SEQ ID NO:3;
iv) a polypeptide having an amino acid sequence of SEQ ID NO:4;
v) a polypeptide having an amino acid sequence of SEQ ID NO:5;
vi) a polypeptide having an amino acid sequence of SEQ ID NO:6;
vii) a polypeptide having an amino acid sequence of SEQ ID NO:11;
viii) a polypeptide having an amino acid sequence of SEQ ID NO:12;
ix) a polypeptide having an amino acid sequence of SEQ ID NO:13; and
x) a polypeptide having an amino acid sequence of SEQ ID NO:14.

Preferably, said antigen is secreted from said organism. Alternatively, said antigen is expressed as a membrane-bound form on the surface of said organism.

Preferably, said recombinant Mycobacterium bovis is a BCG organism.

In a further aspect, the invention provides an Escherichia coli-Mycobacterium bovis shuttle vector comprising a nucleic acid encoding at least an antigenically distinct part of one of one or more Chlamydophila pneumoniae antigens selected from the group consisting of: CopN, CPAF, ADP/ATP Translocase I, SmpB, pmpD, MOMP, LcrH1, LcrH2, OMP2 and HSP60

Preferably, said Escherichia coli-Mycobacterium bovis shuttle vector comprises one or more nucleic acid molecules selected from the group consisting of:
i) a nucleic acid molecule comprising a nucleotide sequence having at least 70% identity to a nucleotide sequence of SEQ ID NO:15;
ii) a nucleic acid molecule comprising a nucleotide sequence having at least 70% identity to a nucleotide sequence of SEQ ID NO:16;
iii) a nucleic acid molecule comprising a nucleotide sequence having at least 70% identity to a nucleotide sequence of SEQ ID NO:17;
iv) a nucleic acid molecule comprising a nucleotide sequence having at least 70% identity to a nucleotide sequence of SEQ ID NO:18;
v) a nucleic acid molecule comprising a nucleotide sequence having at least 70% identity to a nucleotide sequence of SEQ ID NO:19;
vi) a nucleic acid molecule comprising a nucleotide sequence having at least 70% identity to a nucleotide sequence of SEQ ID NO:20;
vii) a nucleic acid molecule comprising a nucleotide sequence having at least 70% identity to a nucleotide sequence of SEQ ID NO:21;
viii) a nucleic acid molecule comprising a nucleotide sequence having at least 70% identity to a nucleotide sequence of SEQ ID NO:22;
ix) a nucleic acid molecule comprising a nucleotide sequence having at least 70% identity to a nucleotide sequence of SEQ ID NO:23; and
x) a nucleic acid molecule comprising a nucleotide sequence having at least 70% identity to a nucleotide sequence of SEQ ID NO:24.

More preferably, said nucleic acid molecule is selected from the group consisting of:
i) a nucleic acid molecule comprising a nucleotide sequence of SEQ ID NO:15;
ii) a nucleic acid molecule comprising a nucleotide of SEQ ID NO:16;
iii) a nucleic acid molecule comprising a nucleotide sequence of SEQ ID NO:17;
iv) a nucleic acid molecule comprising a nucleotide sequence of SEQ ID NO:18;
v) a nucleic acid molecule comprising a nucleotide sequence of SEQ ID NO:19;
vi) a nucleic acid molecule comprising a nucleotide sequence of SEQ ID NO:20;
vii) a nucleic acid molecule comprising a nucleotide of SEQ ID NO:21;
viii) a nucleic acid molecule comprising a nucleotide sequence of SEQ ID NO:22;
ix) a nucleic acid molecule comprising a nucleotide sequence of SEQ ID NO:23; and
x) a nucleic acid molecule comprising a nucleotide sequence of SEQ ID NO:24.

In a further embodiment, the Escherichia coli-Mycobacterium bovis shuttle vector according to comprises a nucleic acid molecule which encodes a polypeptide selected from the group consisting of:
i) a polypeptide having an amino acid sequence at least 70% identical to SEQ ID NO:1;
ii) a polypeptide having an amino acid sequence at least 70% identical to SEQ ID NO:2;
iii) a polypeptide having an amino acid sequence at least 70% identical to SEQ ID NO:3;
iv) a polypeptide having an amino acid sequence at least 70% identical to SEQ ID NO:4;
v) a polypeptide having an amino acid sequence at least 70% identical to SEQ ID NO:5;
vi) a polypeptide having an amino acid sequence at least 70% identical to SEQ ID NO:6;
vii) a polypeptide having an amino acid sequence at least 70% identical to SEQ ID NO:11;
viii) a polypeptide having an amino acid sequence at least 70% identical to SEQ ID NO:12;
ix) a polypeptide having an amino acid sequence at least 70% identical to SEQ ID NO:13; and
x) a polypeptide having an amino acid sequence at least 70% identical to SEQ ID NO:14.

Preferably, said nucleic acid molecule encodes a polypeptide selected from the group consisting of:
i) a polypeptide having an amino acid sequence of SEQ ID NO:1;
ii) a polypeptide having an amino acid sequence of SEQ ID NO:2;
iii) a polypeptide having an amino acid sequence of SEQ ID NO:3;
iv) a polypeptide having an amino acid sequence of SEQ ID NO:4;
v) a polypeptide having an amino acid sequence of SEQ ID NO:5;
vi) a polypeptide having an amino acid sequence of SEQ ID NO:6;
vii) a polypeptide having an amino acid sequence of SEQ ID NO:11;
viii) a polypeptide having an amino acid sequence of SEQ ID NO:12;
ix) a polypeptide having an amino acid sequence of SEQ ID NO:13; and
x) a polypeptide having an amino acid sequence of SEQ ID NO:14.

In a further aspect, the invention provides a Mycobacterium bovis cell transformed or transfected with an Escherichia coli-Mycobacterium bovis shuttle vector as herein described.

In a further aspect, the invention provides a vaccine comprising the recombinant Mycobacterium bovis as herein described, the Mycobacterium bovis cell as herein described or the Escherichia coli-Mycobacterium bovis shuttle as herein described, wherein optionally the vaccine further comprises a pharmaceutically acceptable adjuvant.

In a further aspect, the invention provides use of the recombinant Mycobacterium as herein described, the Mycobacterium bovis cell as herein described or the Escherichia coli-Mycobacterium bovis shuttle vector as herein described for immunizing a subject against a pathogenic microbe of the genus Chlamydophila.

In a further aspect, the invention provide use of the recombinant Mycobacterium bovis as herein described, the Mycobacterium bovis cell as herein described or the Escherichia coli-Mycobacterium bovis shuttle vector as herein described for treating, preventing or reducing a Chlamydophila pneumoniae infection.

In a further aspect, the invention provides use of the recombinant Mycobacterium bovis as herein described, the Mycobacterium bovis cell as herein described or the Escherichia coli-Mycobacterium bovis shuttle vector as herein described for the manufacture of a medicament for immunizing a subject against a pathogenic microbe of the genus Chlamydophila.

Preferably, said pathogenic microbe is Chlamydophila pneumoniae.

In a further aspect, the invention provides use of the recombinant Mycobacterium bovis as herein described, the Mycobacterium bovis cell as herein described or the Escherichia coli-Mycobacterium bovis shuttle vector as herein described for the manufacture of a medicament treating, preventing or reducing a Chlamydophila pneumoniae infection.

In a further aspect, the invention provides a method of immunizing a subject against a pathogenic microbe of the genus Chlamydophila comprising administering a therapeutically effective amount of a vaccine as herein described to a subject.

In a further aspect, the invention provides a method of treating, preventing or reducing a Chlamydophila pneumoniae infection comprising administering a therapeutically effective amount of a vaccine according as herein described to a subject.

Preferably said pathogenic microbe is Chlamydophila pneumoniae.

In a further aspect, the invention provides a recombinant Mycobacterium bovis as herein described for use to prevent, treat or reduce pneumoniae associated with Chlamydophila pneumoniae infection.

In a further aspect, the invention provides a Mycobacterium bovis cell as described herein for use to prevent, treat or reduce pneumoniae associated with Chlamydophila pneumoniae infection.

In a further aspect the invention provide an Escherichia coli-Mycobacterium bovis shuttle vector as described herein, for use to prevent, treat or reduce pneumonia associated with Chlamydophila pneumoniae infection.

In a further aspect, the invention provides a composition comprising the recombinant Mycobacterium bovis as described herein, the Mycobacterium bovis cell as described herein or the Escherichia coli-Mycobacterium bovis shuttle vector as described herein for use to treat, prevent or reduce pneumonia associated with Chlamydophila pneumoniae infection.

In an embodiment of the present invention, there is provided a product e.g. a vaccine for use to treat, inhibit, reduce or prevent atherogenesis, atheroprogression, atherosclerosis, and/or vascular inflammation in a patient. In one embodiment, the vaccine is for the prevention of atheroma formation. In one embodiment, the vaccine is for the prevention of atherosclerosis.

In one embodiment, the vaccine is for preventing the formation of atherosclerotic lesions. In one embodiment, the atherosclerotic lesion may be e.g. a Type I lesion (an initial lesion), a Type II lesion (e.g. a lesion which consists primarily of layers of macrophage foam cells and lipid-laden smooth muscle cells), a Type III lesion (which additionally include collections of extracellular lipid droplets), a Type IV lesion (which are often characterised by a lipid core) also referred to as atheroma), a Type V lesion or a Type VI lesion.

In one embodiment, the vaccine is for retarding or preventing the formation of atheroma e.g. preventing the progression of initial lesions e.g. Type I, Type II or Type III lesions into atheromas, which are classified as Type IV lesions according to Stary et al Arterio, Thromb, & Vasc. Biol, 1995; 15: 1512-1531.

Thus, in one embodiment, the formation of atheroma may be reduced or prevented. The term “atheroma” as used herein includes the formation of an atherosclerotic lesion which is potentially symptom producing e.g. an “advanced” atherosclerotic lesion.

In one embodiment, the vaccine is for the prevention of a cardiovascular event which is associated with atherosclerosis e.g. atheroma formation. In one embodiment, the cardiovascular event is associated with the rupture or fissure of an atheroma. In one embodiment, the cardiovascular event is a disorder selected from the group consisting of thrombosis, myocardial infarction, stroke, transient ischemic attack, occlusive peripheral vascular disease, occlusion of a peripheral artery and complications thereof.

In one embodiment, the vaccine of the present invention may be used to prevent or reduce the risk of e.g. venous thrombosis (e.g. DVT), pulmonary embolism, arterial thrombosis (e.g. in myocardial infarction, unstable angina, thrombosis-based stroke and peripheral arterial thrombosis), systemic embolism usually from the atrium during arterial fibrillation or from the left ventricle after transmural myocardial infarction, or caused by congestive heart failure; prophylaxis of re-occlusion (ie thrombosis) after thrombolysis, percutaneous trans-luminal angioplasty (PTA) and coronary bypass operations; the prevention of re-thrombosis after microsurgery and vascular surgery in general.

Also provided is a method of vaccinating a subject against atheroma comprising administering to a subject a therapeutically effective dose of a vaccine composition as herein described and a method of eliciting an immune response against atheroma comprising administering to an animal or human an immunologically effective dose of a vaccine composition as herein described.

In one aspect of the present invention, there is provided a method of preventing or reducing atherosclerosis in a subject comprising administrating a therapeutically effective amount of a vaccine as described herein to a subject. The present invention also provides a method of preventing or reducing atheroma formation comprising administering a vaccine as described herein to a subject.

In one aspect of the present invention there is provided a vaccine for use in preventing or reducing atherosclerosis wherein the vaccine comprises a recombinant organism as described herein. In one aspect of the present invention, there is provided a vaccine for use in preventing or reducing atheroma wherein the vaccine comprises a recombinant organism as described herein.

The vaccine of the present invention may also further comprise a pharmaceutically acceptable adjuvant, diluent or carrier. Adjuvants may be included in the vaccine to enhance the immune response in the subject. Such adjuvants include, for example, aluminum hydroxide, aluminum phosphate, Freund's Incomplete Adjuvant (FCA), liposomes, ISCOM, and the like. The vaccine may also include additives such as buffers and preservatives to maintain isotonicity, physiological pH and stability. Parenteral and intravenous formulations of the vaccine may include an emulsifying and/or suspending agent, together with pharmaceutically-acceptable diluents to control the delivery and the dose amount of the vaccine.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

The invention will be described in further detail, with reference to the drawings.

FIG. 1: map of LcrE (CpB0334) inserted into vector pET-11a.

FIG. 2: control digestion of LcrE-pET and LcrE-pMV vectors.

FIG. 3: results of LcrE purification (SDS PAGE).

FIG. 4 shows the level of LcrE-specific IgG antibodies in mouse sera after one, two and three C. pneumoniae inoculations detected by ELISA.

FIG. 5 shows a map of ADP/ATP translocase (CpB0359) inserted into vector pET-11a.

FIG. 6: ATP/ADP translocase overexpressed in different strains (BL21 and BL21 LysS) and at different cell densities (OD₆₀₀ 0.6, 0.7 and 0.8, SDS PAGE).

FIG. 7: map of MOMP (CpB0722) in plasmid pGEM T Easy.

FIG. 8: A: Map of SmpB insert and pET vector; B: SDS PAGE of SmpB overexpression in E. coli strain BL21 LysS at different cell densities (OD₆₀₀ 0.6, 0.7 and 0.8). NI=non-induced.

FIG. 9: Lung weight in mice 3, 7 and 28 days after challenge with C. pneumoniae A: after intranasal BCG immunisation; B: after subcutaneous BCG immunisation.

FIG. 10: C. pneumoniae titre in the lungs of mice challenged with C. pneumoniae strains TWAR or CV6; A: after intranasal BCG immunisation; B: after subcutaneous BCG immunisation.

FIG. 11: A: C. pneumoniae-specific IgG titre in the sera of C. pneumoniae-infected naïve and BCG-immunised mice; B: cHSP60-specific antibodies in pre-challenge sera and in sera 4 weeks after TWAR or CV-6 challenge.

FIG. 12: C. pneumoniae-specific immunoglobulin in lungs of BCG-immunised and naïve individual mice challenged with TWAR or CV-6; A: IgG; B: IgA.

FIG. 13: A: Proliferation of spleen cells stimulated with C. pneumoniae or mock antigen; B: IFN-γ level in lungs of BCG-immunised and C. pneumoniae TWAR-challenged mice.

FIG. 14: IL-6 levels in the lungs of BCG-immunised and C. pneumoniae TWAR-challenged mice.

FIG. 15: A: Protection against C. pneumoniae infection as measured by culture of C. pneumoniae from the lungs of LcrE protein-immunized mice.

FIG. 16: LcrE-specific IgG subtypes: IgG1 and IgG2a in LcrE protein-immunized mouse sera after C. pneumoniae infection.

FIG. 17: The mucosal immunity was tested by demonstration of IgA in the sera and in the lungs.

FIG. 18: Local cytokine levels (IL-4, IL-6, IL-10, IFN-γ) in the supernatant of lungs of LcrE-immunized mice after C. pneumoniae infection.

FIG. 19: Proliferation indices measured in three individual mice.

FIG. 20: IL-4 was only produced by LcrE+Alum immunized mice in response to LcrE stimulation.

FIG. 21: IL-10 was secreted only by the spleen cells of LcrE+Alum immunized mice in response to LcrE stimulation. C. pneumoniae stimulation induced IL-10 production also in immunized and in non-immunized mice.

FIG. 22: C. pneumoniae stimulation induced IL-6 production in all groups at comparable level. LcrE-stimulation resulted in IL-6 production in non-immunized, but C. pneumoniae-infected mice suggesting an LcrE-inducible component of C. pneumoniae infection evoked cellular response. Previous LcrE immunization resulted in an increased IL-6 production.

FIG. 23: IFN-γ was not produced in non-immunized mice in response to LcrE stimulation, but LcrE-immunization induced IFN-γ producing cells. The level of IFN-γ after C. pneumoniae stimulation was not different in non-immunized and LcrE-immunized groups.

FIG. 24: A,B ELISOT assay was sensitive enough to reveal the presence of LcrE-specific IFN-γ producing cells among the spleen cells of non-immunized C. pneumoniae infected mice. The number of LcrE-specific IFN-γ producing cells was higher in LcrE-immunized mice than in non-immunized mice. Further tests with the frozen cells of other mice are needed to compare the effect of Freund's and Alum adjuvant on the number of IFN-γ producing cells.

FIG. 25: A, B Phenotype of LcrE-specific and viable C. pneumoniae induced IFN-γ producing spleen cells is CD4⁺ as determined by ELISPOT assay after removal of CD4⁺ or CD8⁺ T cells.

FIG. 26: LcrE (chlamydial outer protein N-CopN, gene: pCB0334) and MOMP (major outer membrane protein, gene: CpB0722) Primers (U1, U2) that are universal for amplification of inserted genes from pET based constructs are used to amplify and transfer genes into pJH154 secreting mycobacterial plasmid.

### DETAILED DESCRIPTION

The present invention relates to E. coli-BCG shuttle plasmids expressing C. pneumoniae antigens capable of inducing protective immune responses against C. pneumonia infection.

A number of candidate vaccines based on BCG expressing secreted recombinant proteins have been developed, including rBCG secreting soluble pneumococcal surface protein A (PspA) (Langermann et al 1994, J Exp Med 180: 2277-2286); and Plasmodium merozoite surface protein 1 (MSP1) (Matsumoto et al, 1998 J Exp Med 188: 845-854). In addition to expressing directly immunogenic proteins, rBCG have been constructed that secrete immunostimulatory factors such as IL-2 (He et al, 2002, Int J Oncology 20: 1305-1311) in combination with either surface-bound or soluble immunogens. The usual secretion mechanism used is based on the secreted mycobacterial α-antigen (α-B) (Matsuo et al, 1990, Infection and Immunity, 58: 4049-4054).

The inventors have introduced (subcutaneously or intranasally) into mice recombinant BCG-s expressing C. pneumoniae antigens. Immunogenicity is then monitored and protective efficacy evaluated after intranasal C. pneumoniae challenge with respiratory strain TWAR and CV-6 cardiovascular strain. The CV-6 strain which was isolated from the coronary artery of a bypass surgery patient may have a pronounced capability for systemic dissemination.

A number of potentially antigenic Chlamydophila pneumoniae proteins are known or predicted from genomic sequences.

Low Calcium Response E (LcrE), also known as Chlamydial outer membrane protein N (CopN) or type III secreted protein SctW (Ordered Locus Name CpB0334, TrEMBL accession number Q9Z8L4), is a component of a Type III secretion system (TTSS) responsible for the export of bacterial proteins into the host cell cytosol (Slepenkin et al, 2005, J Bacteriol 187: 473). It shows homology with yopN from Yersinia, and invE from Salmonella and has been shown to be capable of generating a protective immune response against C pneumoniae in mice (Thorpe et al 2007, Vaccine 25: 2252). A type III secretion system constituting protein (putative “lid” of the TTS), localised mainly in the inclusion membrane, and also in EB, The polypeptide sequence of LcrE is shown in SEQ ID NO:1. The nucleic acid sequence that encodes LcrE is shown in SEQ ID NO:15.

Chlamydial protease activity-like factor (CPAF), also known as CPn_{—}1016 (Ordered Locus Name CpB 1054, TrEMBL accession number Q9Z6P3) is a poorly-characterised 619 amino acid protein (unprocessed precursor). CPAF is a Chlamydial protease-like activity factor for evading host defense. It is believed to be secreted into the host cell cytosol for degrading host transcription factors required for major histocompatibility complex antigen expression. It degrades RFX5, a transcription factor required for MHC antigen expression. The polypeptide sequence of CPAF is shown in SEQ ID NO:3. The nucleic acid sequence that encodes CPAF is shown in SEQ ID NO:17.

ADP/ATP translocase 1, also known as tIcA (Ordered Locus Name CpB0359, TrEMBL accession number Q9Z8J2) is one of a group of conserved ADP/ATP translocases common to many obligate intracellular organisms such as Chlamydiae (Schmitz-Esser et a/2004 J Bacteriol 186: 683). ADP/ATP translocase is an energy provider by host ATP-chlamydial ADP exchange. The polypeptide sequence of ADP/ATP translocase I_s shown in SEQ ID NO:2. The nucleic acid sequence that encodes ADP/ATP translocase I is shown in SEQ ID NO:16.

Major outer membrane porin (MOMP), also known as ompA (Ordered Locus Name CpB0722, TrEMBL accession number P27455), is a multi-transmembrane domain protein forming part of a disulphide-linked outer membrane complex together with the small cysteine-rich protein (omcA) and the large cysteine-rich periplasmic protein (omcB). The polypeptide sequence of MOMP is shown in SEQ ID NO:5. The nucleic acid sequence that encodes MOMP is shown in SEQ ID NO:19.

Small protein B (SmpB), also known as SsrA-binding protein (Ordered Locus Name CpB0345, TrEMBL accession number Q9Z8K₁) binds specifically with transfer mRNA (SsrA or 10Sa RNA) and is required for the stable association of transfer mRNA with ribosomes (Dulebohn et al, 2006, J Blol Chem 281: 28536). The polypeptide sequence of SmpB is shown in SEQ ID NO:6. The nucleic acid sequence that encodes SmpB is shown in SEQ ID NO:20.

Probable Outer Membrane Protein 13 (pmp13) Precursor (pmpD), also known as Outer membrane protein 14 (OMP 14) and polymorphic membrane protein 13 (Ordered Locus Name CpB0470, TrEMBL accession number Q9Z896) is a poorly characterised putative outer membrane protein. PmpD is a member of the polymorphic membrane protein family, serves as adhesin, is involved in molecular transport and signalling. The polypeptide sequence of pmpD is shown in SEQ ID NO:4. The nucleic acid sequence that encodes pmpD is shown in SEQ ID NO:18.

Low Calcium Response Protein H 1 (LCRH1) TrEMBL accession number NP_{—}225006. A TTS constituting proteins-chaperone, LcrH1 is expressed late in the developmental cycle, IFN-γ abolishes its expression. The polypeptide sequence of LcrH1 is shown in SEQ ID NO:11. The nucleic acid sequence that encodes LcrH1 is shown in SEQ ID NO:21.

Low Calcium Response Protein H 2 (LcrH2) TrEMBL accession number NP_{—}225215. A TTS constituting proteins-chaperone, LcrH2 is expressed throughout the developmental cycle, unaffected by IFN-γ. The polypeptide sequence of LcrH2 is shown in SEQ ID NO:12. The nucleic acid sequence that encodes LcrH2 is shown in SEQ ID NO:22.

Outer Membrane Protein 2 (OMP2) is a 60K cysteine-rich outer membrane protein precursor. TrEMBL accession number NP_{—}876851. OMP2 is abundant protein on EB surface, has a role in differentiation of RB into EB and carries genus specific epitopes. The polypeptide sequence of OMP2 is shown in SEQ ID NO:13. The nucleic acid sequence that encodes OMP2 is shown in SEQ ID NO:23.

Chaperonin HSP60 TrEMBL accession number NP_{—}877201. The polypeptide sequence of HSP60 is shown in SEQ ID NO:14. The nucleic acid sequence that encodes HSP60 is shown in SEQ ID NO:24.

In accordance with the invention the aforementioned genes are amplified by PCR and cloned into pMV262 or pJH154 E. coli-BCG shuttle plasmids, and electroporated into a BCG Mycobacterium bovis organism. The transformants are screened for the presence of the plasmid and Western blot will be used for assessing the expression of the C. pneumoniae antigen. C. pneumoniae strain TWAR and cardiovascular strain CV-6 are propagated in Hep2 cells and concentrated by high speed centrifugation to produce antigen used in ELISA test and as inoculum for intranasal infection of mice.

In one embodiment, the present invention provides a vector which comprises a C. pneumoniae nucleic acid as described above. As used herein, a “vector” may be any of a number of nucleic acids into which a desired sequence may be inserted. Vectors include, but are not limited to, plasmids, phagemids and virus genomes. A cloning vector is one which is able to replicate in a host cell, and which typically is further characterized by one or more endonuclease restriction sites at which the vector may be cut in a determinable fashion and into which a desired DNA sequence may be ligated such that the recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

Vectors may further contain one or more selectable marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., p-galactosidase, luciferase), and genes which visibly affect the phenotype of transformed or transfected cells, hosts, colonies or plaques (e.g., various fluorescent proteins such as green fluorescent protein, GFP). Preferred vectors are those capable of autonomous replication, also referred to as episomal vectors. Alternatively vectors may be adapted to insert into a chromosome, so called integrating vectors. The vector of the invention is typically provided with transcription control sequences (promoter sequences) which mediate cell/tissue specific expression. These promoter sequences may be cell/tissue specific, inducible or constitutive.

Promoter is a term recognised in the art and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues which include, by example and not by way of limitation, intermediary metabolites, environmental effectors.

Promoter elements also include so called TATA box, RNA polymerase initiation selection (RIS) sequences and CAAT box sequence elements which function to select a site of transcription initiation. These sequences also bind polypeptides which function, inter alia, to facilitate transcription initiation selection by RNA polymerase.

Adaptations also include the-provision of autonomous replication sequences which both facilitate the maintenance of said vector in either the eukaryotic cell or prokaryotic host, so called “shuttle vectors”. Vectors which are maintained autonomously are referred to as episomal vectors. Episomal vectors are desirable since these molecules can incorporate large DNA fragments (30-SOkb DNA). In one embodiment, the vector is suitable for use in a Mycobacterium bovis organism e.g. a BCG M. bovis organism.

### DEFINITIONS

Antigens: As used herein the term “antigens” relates to molecules that interact with specific lymphocyte receptors-surface T cell antigen receptors and B cell immunoglobulin receptors. A particular B or T cell binds to a very specific region of the antigen, called an antigenic determinant or epitope.

Vaccines_As used herein the term “vaccine” relates to a composition used to vaccinate an animal that contains at least one proteinaceous agent that induces the stimulation of the host immune system and prevents or attenuates subsequent unwanted pathology associated with the host reactions to subsequent exposures of the pathogen.

The vaccine may comprise an adjuvant and or carrier. An adjuvant is a substance or procedure which augments specific immune responses to antigens by modulating the activity of immune cells. Examples of adjuvants include, by example only, agonsitic antibodies to co-stimulatory molecules, Freunds adjuvant, muramyl dipeptides, liposomes. An adjuvant is therefore an immunomodulator. A carrier is an immunogenic molecule which, when bound to a second molecule augments immune responses to the latter.

Treatment As used herein, the terms “treatment”, “treating” and the like generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject, such as mammal, particularly a human, and includes: (a) preventing the disease from occurring in a subject which may be predisposed to the disease; (b) inhibiting the disease, i.e. arresting its development; or (c) relieving the disease, i.e. causing regression of the disease. The term “treatment” may also include alleviating symptoms of a disorder.

Immunize_As used herein, the term “immunize” is defined as eliciting an immune response in an subject, such as an animal, both a humoral immune response and a cellular immune response.

Nucleic Acid As used herein, the term “nucleic acid molecule” and “nucleic acid” includes DNA molecules (e.g., a cDNA or genomic DNA) and RNA molecules (e.g., a mRNA) and analogs of the DNA or RNA generated, e.g., by the use of nucleotide analogs. The nucleic acid molecule can be single-stranded or double-stranded, but preferably is double-stranded DNA.

The invention includes a nucleic acid sequence that is the complement of the nucleotide sequences shown in any of SEQ ID NO's:15, 16, 17, 18, 19, 20, 21, 22, 23 or 24, or portions or fragments thereof. In other embodiments, the invention includes a nucleic acid sequence that is sufficiently complementary to the nucleotide sequence shown in any of SEQ ID NO's: 15, 16, 17, 18, 19, 20, 21, 22, 23 or 24 such that it can hybridize to the nucleotide sequences shown in any of SEQ ID NO's:15, 16, 17, 18, 19, 20, 21, 22, 23 or 24 respectively, thereby forming stable duplexes.

As used herein, the term “hybridizes under stringent conditions” describes conditions for hybridization and washing. Stringent conditions are known to those skilled in the art and can be found in available references (e.g., Current Protocols in Molecular Biology, John Wiley & Sons, N.Y., 1989, 6.3.1-6.3.6). Aqueous and non-aqueous methods are described in that reference and either can be used. A preferred example of stringent hybridization conditions are hybridization in 6× sodium chloride/sodium citrate (SSC) at about 45° C., followed by one or more washes in 0.2×SSC, 0.1% (w/v) SDS at 50° C. Another example of stringent hybridization conditions are hybridization in 6×SSC at about 45° C., followed by one or more washes in 0.2×SSC, 0.1% (w/v) SDS at 55° C. A further example of stringent hybridization conditions are hybridization in 6×SSC at about 45° C., followed by one or more washes in 0.2×SSC, 0.1% (w/v) SDS at 60° C. Preferably, stringent hybridization conditions are hybridization in 6×SSC at about 45° C., followed by one or more washes in 0.2×SSC, 0.1% (w/v) SDS at 65° C. Particularly preferred stringency conditions (and the conditions that should be used if the practitioner is uncertain about what conditions should be applied to determine if a molecule is within a hybridization limitation of the invention) are 0.5 molar sodium phosphate, 7% (w/v) SDS at 65° C., followed by one or more washes at 0.2×SSC, 1% (w/v) SDS at 65° C. Preferably, the invention includes nucleic acid molecules that hybridizes under stringent conditions to the sequence of SEQ ID NO:15, 16, 17, 18, 19, 20, 21, 22, 23 or 24 corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

The invention includes nucleic acid sequences which encode a naturally occurring allelic variant of a polypeptide comprising the amino acid sequence shown in SEQ ID NO: 1, 2, 3, 4, 5, 6, 11, 12, 13, 14. Functional allelic variants will typically contain only conservative substitution of one or more amino acids of SEQ ID NO: 1, 2, 3, 4, 5, 6, 11, 12, 13, 14 or substitution, deletion or insertion of non-critical residues in non-critical regions of the protein. Non-functional allelic variants will typically contain a non-conservative substitution, a deletion, or insertion or premature truncation of the amino acid sequence of SEQ ID NO: 1, 2, 3, 4, 5, 6, 11, 12, 13, 14, or a substitution, insertion or deletion in critical residues or critical regions. Nucleic acid molecules corresponding to natural allelic variants and homologues of the hydrogenase nucleic acid molecules of the invention can be isolated based on their homology to the nucleic acid molecules of the invention using the nucleotide sequences described in SEQ ID NO:15, 16, 17, 18, 19, 20, 21, 22, 23 or 24, or a portion thereof, as a hybridization probe under stringent hybridization conditions.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in protein is preferably replaced with another amino acid residue from the same side chain family. Alternatively, in another embodiment, mutations can be introduced randomly along all or part of coding sequences, such as by saturation mutagenesis, and the resultant mutants can be screened for biological activity to identify mutants that retain activity.

The invention includes nucleic acid sequences that are at least about: 60%, 65%, 70%, 75%, 80%, 85%, 90%, 91%, 92%, 93%, 94%, 95%, 96%, 97%, 98%, 99% or 100%, homologous to the entire length of the nucleotide sequence shown in SEQ ID NO's:15, 16, 17, 18, 19, 20, 21, 22, 23 or 24, or portions or fragments thereof.

The invention includes polypeptides having amino acid sequences that are at least about: 60%, 65%, 70%, 75%, 80%, 85%, 90%, 91%, 92%, 93%, 94%, 95%, 96%, 97%, 98%, 99% or 100%, homologous to the entire length of the polypeptide sequence shown in SEQ ID NO's: 1, 2, 3, 4, 5, 6, 11, 12, 13, 14, or portions or fragments thereof.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

### Example 1

### Expression of ADP/ATP Translocase and LcrE

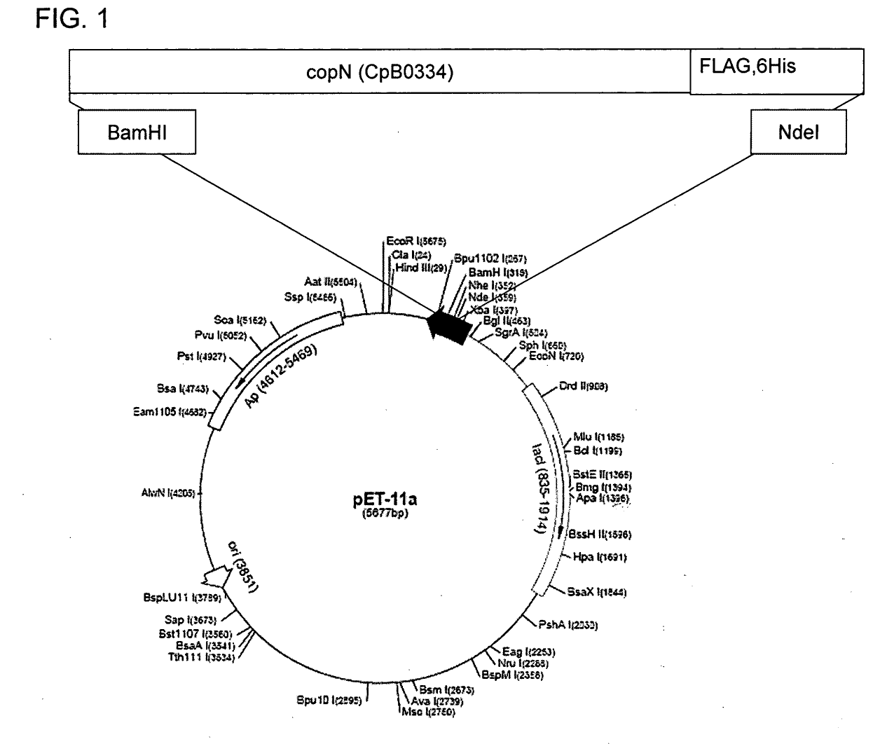

Initially, a Bam HI/Nde I, N-terminally FLAG- and His-tagged, LcrE insert was ligated into a pET-11a expression vector (FIG. 1). E. coli HB101 strain was transformed with the plasmid and expression was tested with induction of gene expression and purification of the 43.4 kDa protein.

Subsequently, a 1.5 kb fragment containing the transporter gene (CpB0359) and a 1.2 kb fragment containing the gene of LcrE (CpB0334) was amplified by PCR, using the oligonucleotide primers:

(SEQ ID NO: 7)

| | |
|---|---|
| A1 | 5′-GTG CAT ATG ACA AAA ACC GAA G-3′, |

(SEQ ID NO: 8)

| | |
|---|---|
| A2 | 5′-GCT GGA TCC ACT AAA AAG CAG GTA G-3′, |

(SEQ ID NO: 9)

| | |
|---|---|
| L1 | 5′-GGA GGC ATA TGG CAG CAT CA-3′ |
| and | |

(SEQ ID NO: 10)

| | |
|---|---|
| L2 | 5′-CAC AGG ATC CGT ATT GGT TTT GCA TGG C-3′ |

with a GeneAmpII (Applied Biosystems) thermocycler with Advantage GC cDNA polymerase (BD Biosciences). PCR amplification conditions were as recommended by the manufacturer. The oligonucleotides were designed by using the complete sequence of the Chlamydophila pneumoniae TW-183 genome [NCBI]. Chlamydophila pneumoniae TW-183 DNA was used as template. The amplified DNA was digested with NdeI and BamHI and inserted into the p6HisF-11d (icl) plasmid (Honer zu Bentrup et al (1999, J Bacteriol 181: 7161) after digesting it with NdeI and BamHI and by replacing the icl gene. The resulting plasmids, pATP1 and pET-L, respectively and a control plasmid with the chlamydial smpB gene (CpB0345) were used to express ATP/ADP transporter and the LcrE protein in E. coli. For overexpression, Escherichia coli HB101 (pGP1-4) cells carrying the pET-L plasmid or E. coli HB101 (pGP1-4), BL21 and BL21 LysS strains carrying the pATP1 were grown and treated according to the method of Tabor and Richardson (1985, Proc. Natl. Acad. Sci. USA 82:1074) or according to the manufacturers instructions. Briefly, E. coli HB101 (pGP1-4) cells containing the plasmids were grown at 32° C. in LB medium in the presence of the required antibiotics. Overexpression of the protein was induced by shifting the temperature to 42° C. for 20 minutes. After induction, the temperature was shifted down to 37° C. for an additional 120 minutes, the cells were harvested by centrifugation and the cell pellets were frozen. E. coli BL21 and BL21LysS strains carrying the pATP1 were induced with 1 mM IPTG for 1 hour.

### Results

The chlamydial genes CpB0359 and CpB0334 were successfully cloned into both pET-based vectors. The ATP/ADP transporter is consisting of 426 amino acids and its molecular mass is 57 kDa. The LcrE protein is consisting of 399 amino acids and its calculated molecular mass is 43.4 kDa. Because of the His and FLAG tags our fusion proteins are 4 kDa larger. The LcrE protein was eluted in all fractions, but it reached the highest concentration in the second fraction (E2), in which its concentration was ˜6 μg/μl. This fraction was used in further serological tests.

### Example 2

### Immunisation with Recombinant LcrE

With the purified LcrE protein Western blot and ELISA tests were carried out using sera of mice inoculated by intranasal route one, two or three times with 1×10⁶ inclusion forming units (IFU) of Chlamydophila pneumoniae. The mouse sera were taken 4 weeks after the last inoculation or in case of three times-inoculated mice 4 and 8 weeks after the last inoculation.

Western blot: 6 μg purified LcrE protein was separated on a 10% SDS-PAGE and blotted. The protein was probed using 50 times diluted mouse sera. Bound antibody was detected with horseradish peroxidase-conjugated goat anti-mouse IgG and the substrate 3,3-diaminobenzidine tetrahydrochloride (DAB).
ELISA: 100 ng/well LcrE protein was used to coat the wells of an ELISA plate. Mouse sera diluted 1/50 were applied to all plate wells and horseradish peroxidase-conjugated goat anti-mouse IgG was added to the wells as secondary antibody. Substrate solution containing hydrogen-peroxide and o-phenylene diamine (OPD) was used for detection.

In silico, the secondary structure and the antigenicity of the LcrE protein and the ATP/ADP transporter were determined.

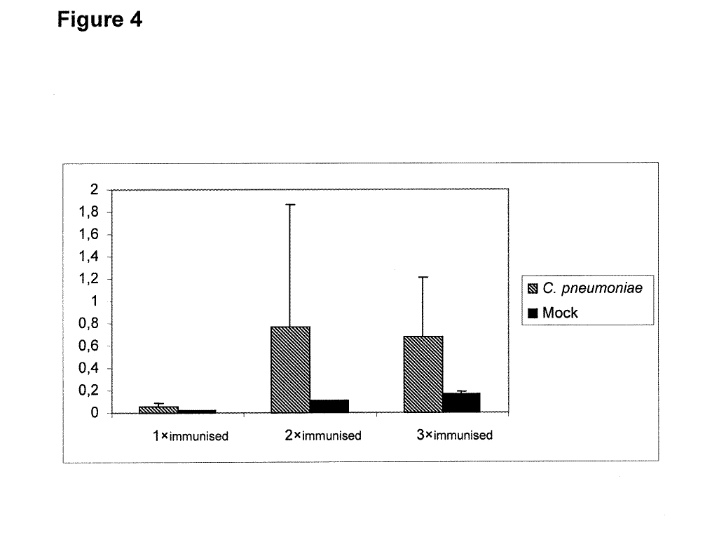

The denatured protein tested with Western blot using three-times Chlamydia-challenged mouse sera failed to give a reaction; whereas with ELISA the native protein gave a reaction with the same mouse sera. This agrees with the findings of Tammiruusu et al (2007, Vaccine 25(2):283-90.). After single inoculation with C. pneumoniae LcrE-specific antibody was not detectable with ELISA test (O.D.=0.05±0.04), on the other hand our results show that for reaching detectable level of antibody production mice should be inoculated repeatedly, at least two times (O.D.=0.77±1.10), or three times (O.D.=0.68±0.53). Persistently high IgG level was detected 8 weeks after the third inoculation (O.D.=1.00±1.27) (FIG. 4).

### Example 3

### Expression of LcrE from BCG Shuttle Vector

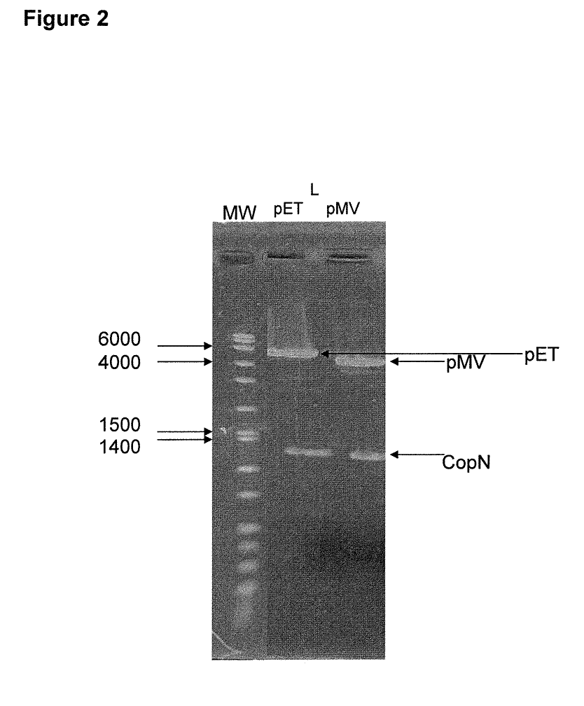

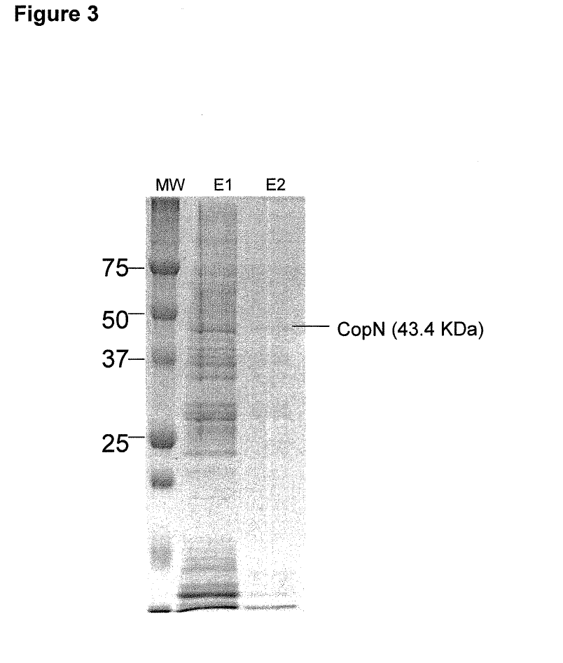

The LcrE insert was excised from pET-L by Bam HI/Nde I digestion and both insert and Bam HI/Nde I-cut pMV262 E. coli-mycobacterium shuttle plasmid were purified from gel slices and ligated (FIG. 2). The resultant BCG shuttle vector pMV-L carries the LcrE insert with the mycobacterial icl promoter (Rv0467) and N-terminal 6His and FLAG tags. The vector (pET-L) was transformed into E. coli DH5a for plasmid purification and was checked for insert. The tagged LcrE was overexpressed in E. coli HB101 transformed with pET-L (FIG. 3).

pMV-L and also a control with the chlamydial smpB were transformed to Mycobacterium bovis BCG by electroporation. Bacteria were grown to the late logarithmic phase in Middlebrook 7H9Broth (Difco) with OADC (oleate-albumin-dextrose complex) enrichment and 0.05% Tween 80, and the cells were harvested by centrifugation.

### Example 4

### Expression of ADP/ATP Translocase I from BCG Shuttle Vector

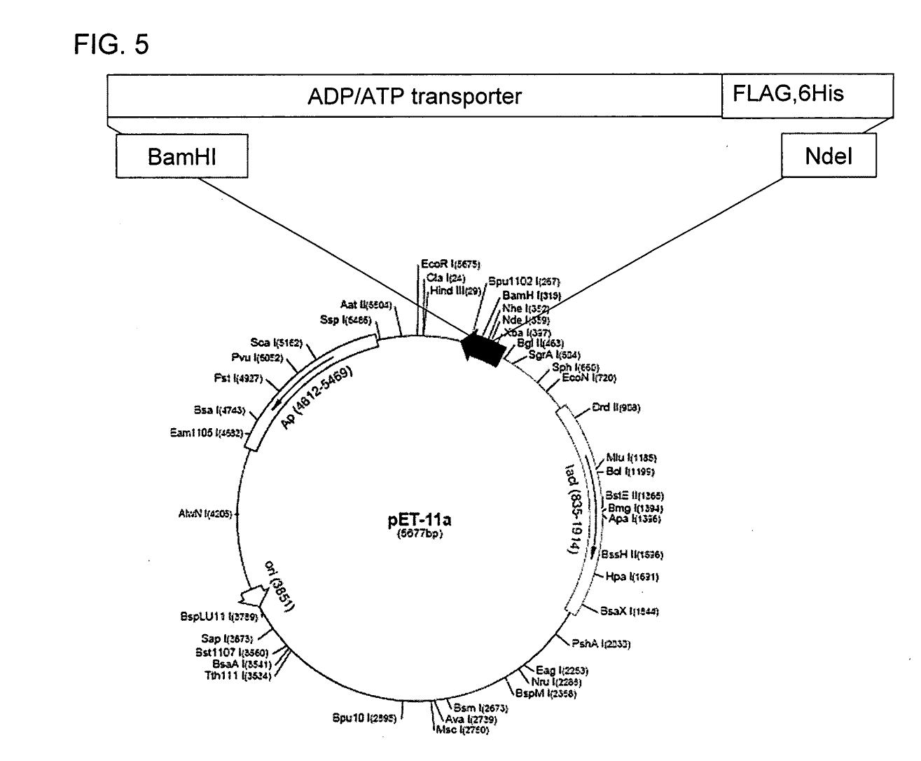

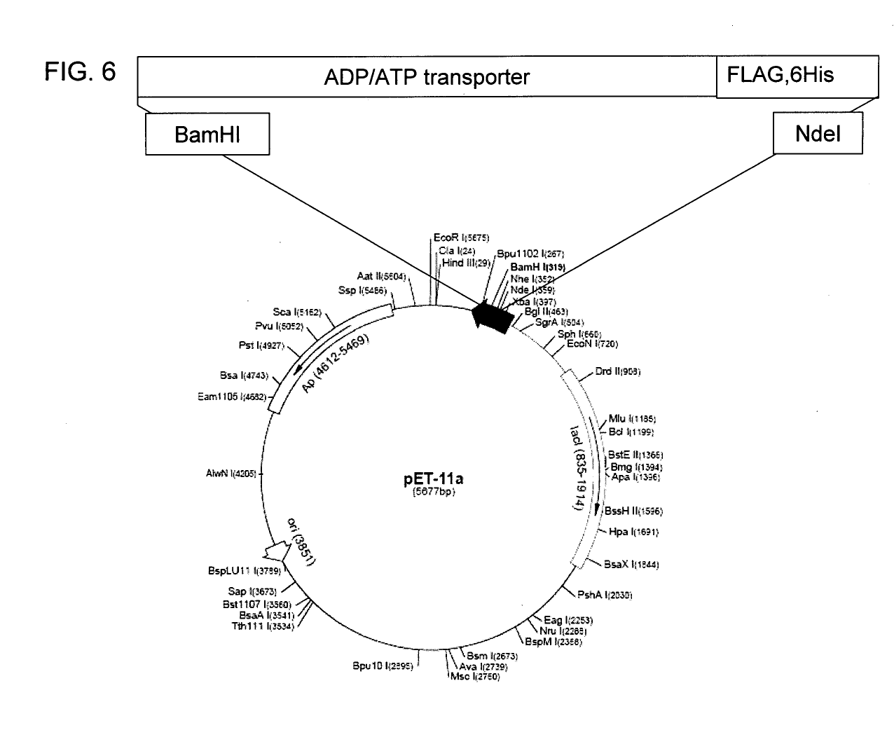

Similarly, a Bam HI/Nde I insert encoding N-terminally FLAG- and His-tagged ADP/ATP translocase was cloned into a pET vector (FIG. 5) to give pATP1 and then into pMV262, to give pATP2 E. Coli HB101, BL21 and BL21 LysS strains were transformed with pATP1. Expression was tested with induction of gene expression and purification of the 57 kDa protein (FIG. 6)

Purification was carried out after overexpression in HB101 for 20 minutes at 42° C.-with His-Select cartridge (Sigma) according to manufacturer's instructions.

Another expression system was tested in E. Coli BL21 and BL21 LysS strains induced with 1 mM IPTG for 1 hour.

### Example 5

### Construction of OMP14, CPAF, MOMP and SmpB Expressing BCG Shuttle Vectors

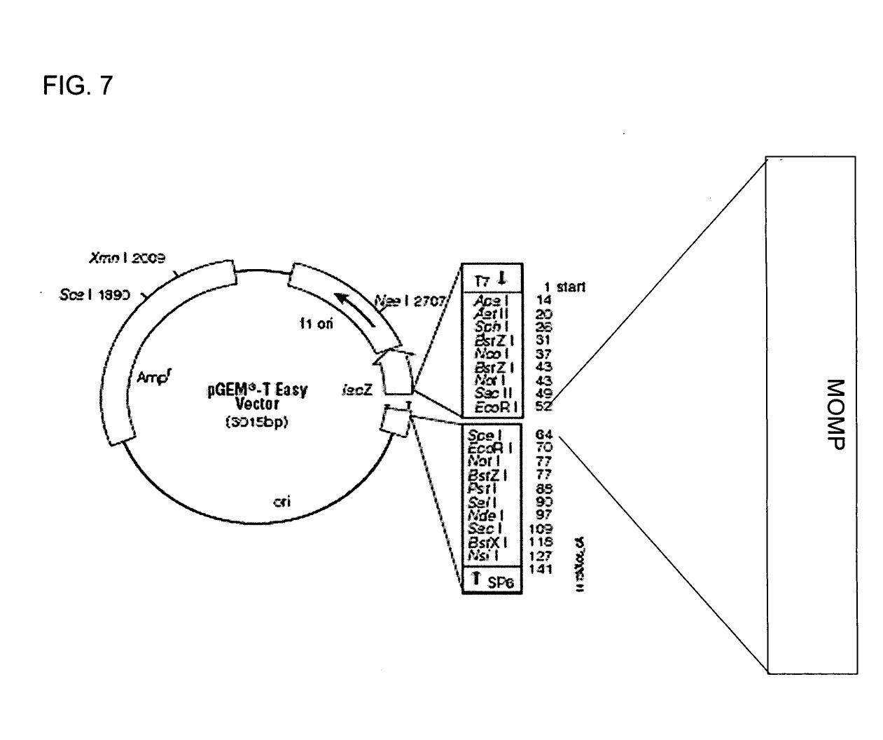

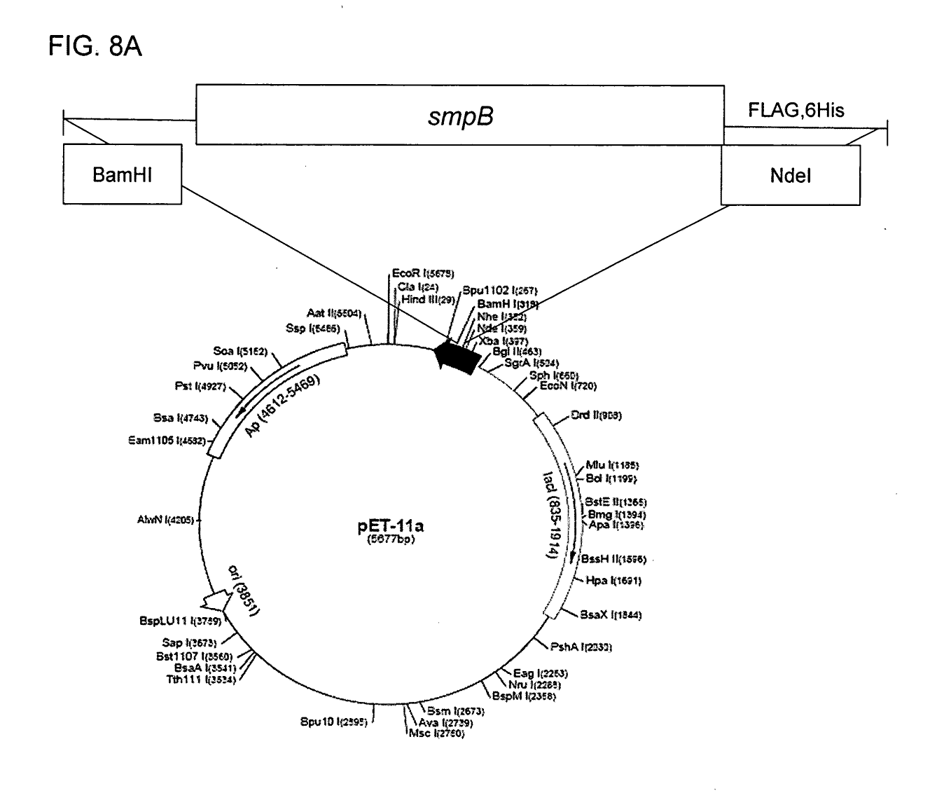

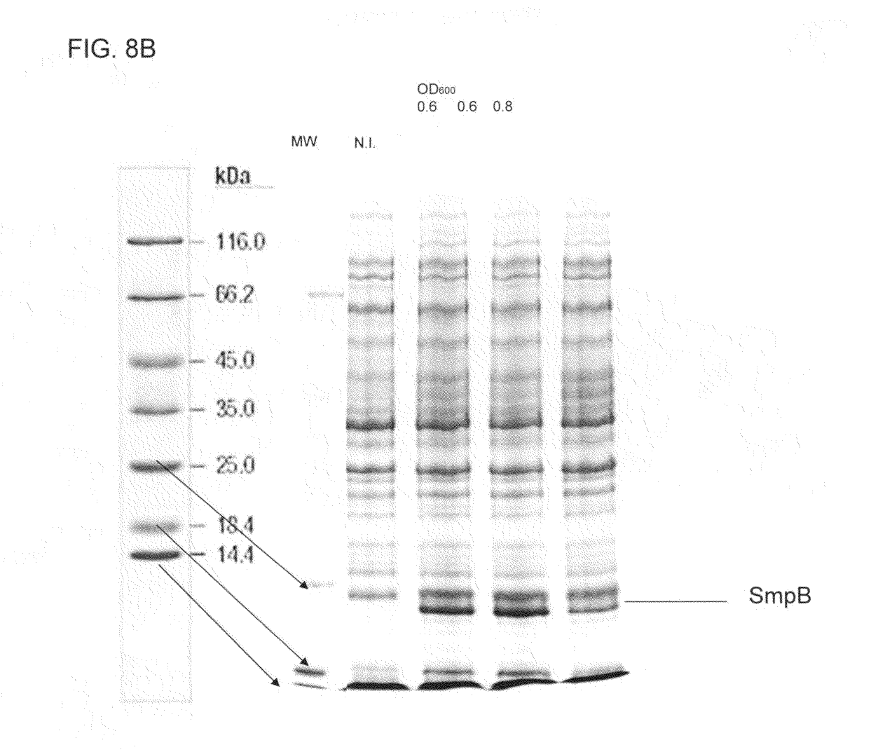

pmpD, CPAF, MOMP (FIG. 7) and SmpB-expressing (FIG. 8) pMV vectors were constructed as described above.

### Example 6

### Immunisation with rBCG Expressing Chlamydial ADP/ATP Translocase

7-8-week old female BALB/c mice immunised twice (8-week interval) intranasally or subcutaneously with parental BCG or recombinant BCG expressing chlamydial ADP/ATP-translocase. Four weeks after the second BCG inoculation at the age of 19-20 weeks, mice received an intranasal challenge inoculation of C. pneumoniae TWAR or C. pneumoniae CV-6 cardiovascular strain.

In a control experiment mice were infected with the same batch and dose of TWAR and CV-6 strains without prior BCG-immunisation. Mice were infected with C. pneumoniae strains at the age of 19-20 weeks.

Samples from C. pneumoniae-inoculated mice were collected 3, 7 and 28 days after inoculation for analysis as follows.
1. Plasma was collected for antibody detection (C. pneumoniae-specific IgG, IgA and chlamydial HSP60 (cHSP60)-specific IgG)
2. Spleens were harvested for testing for C. pneumoniae-specific lymphocyte proliferation as a sign of cellular immune response.
3. Lungs were harvested to estimate extent of inflammation, bacterial burden and local antibody (C. pneumoniae-specific IgG, IgA) and cytokine response.

The experimental groups are summarised in Table 1.

TABLE 1

| Groups | Immunisation | C. pneumoniae challenge | |
|---|---|---|---|
| BCG in | Intranasal, parental BCG | TWAR strain | CV-6 strain |
| ‘ATP’ in | Intranasal, recombinant | TWAR strain | CV-6 strain |
| | BCG-ADP/ATP translocase | | |
| BCG sc | Subcutaneous, parental BCG | TWAR strain | CV-6 strain |
| ‘ATP’ sc | Subcutaneous, recombinant | TWAR strain | CV-6 strain |
| | BCG/-ADP/ATP translocase | | |
| naive | Non-immunised | TWAR strain | CV-6 strain |

### Results

Data from the control experiment (non-immunised mice infected with C. pneumoniae) were combined with the results of the BCG immunization experiment and are presented as follows.

### Lung Inflammation

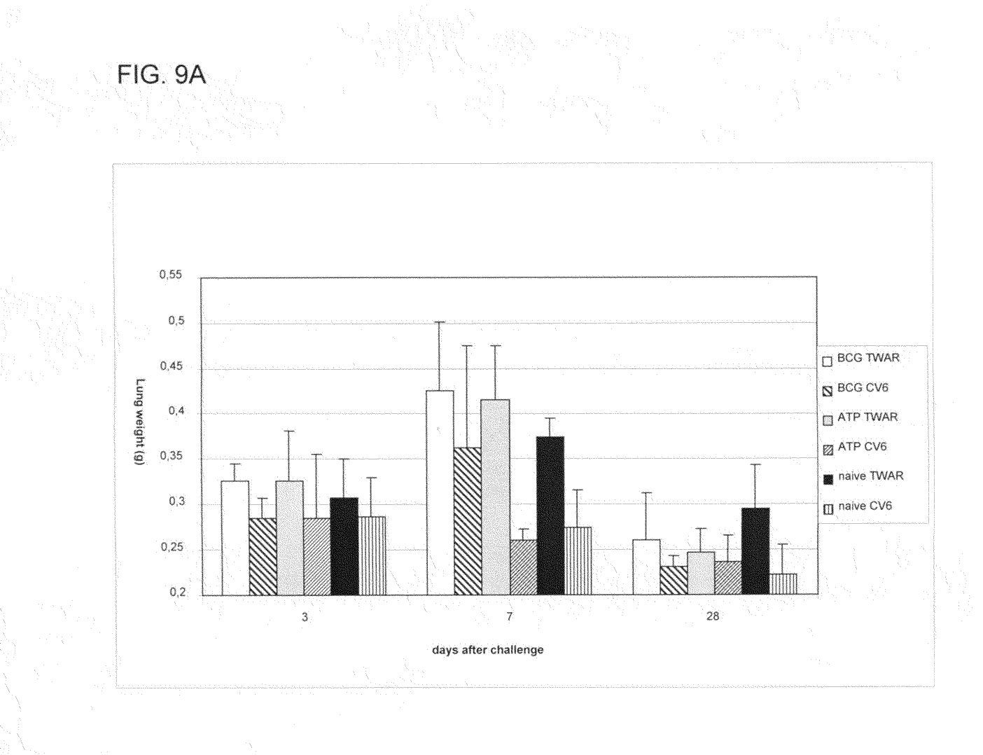

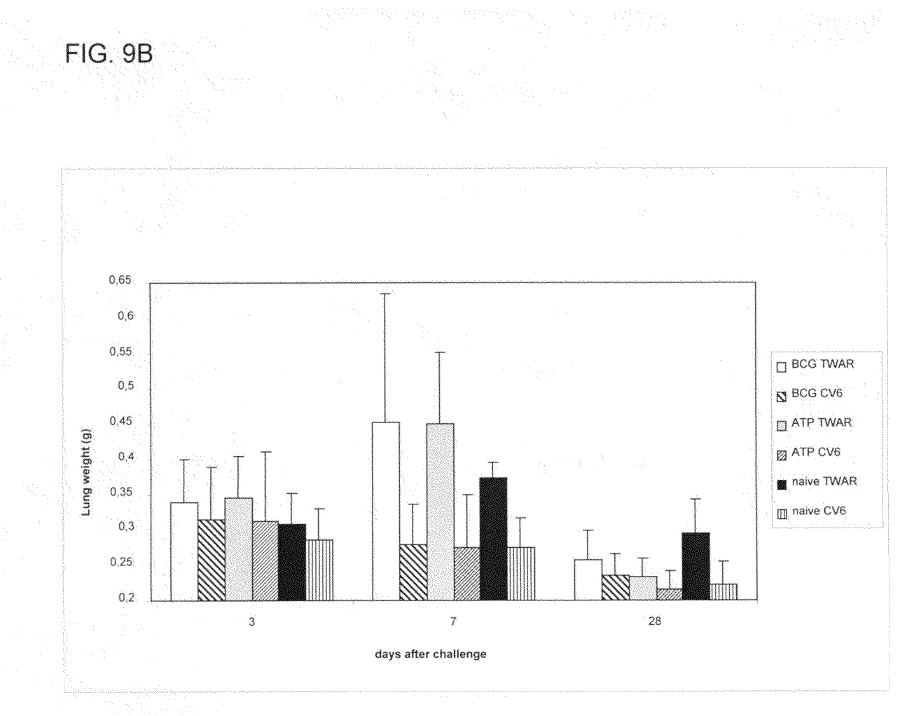

The severity of lung inflammation as reflected by the weight of lungs was compared in different groups after challenge inoculation (FIG. 9). In case of TWAR inoculated non-immunised mice, the weight of lung was lower at days 3 and 7 than in BCG-immunised mice, suggesting that prior BCG-immunization might increase the severity of inflammation. This phenomenon was not observed in CV-6 inoculated mice. In TWAR-inoculated mice the level of lung inflammation peaked on day 7 after challenge, while in CV-6 inoculated group lung weights decreased from the day 3 time point similarly to the mice with prior BCG-immunisation.

Infective C. pneumoniae Lung Titre

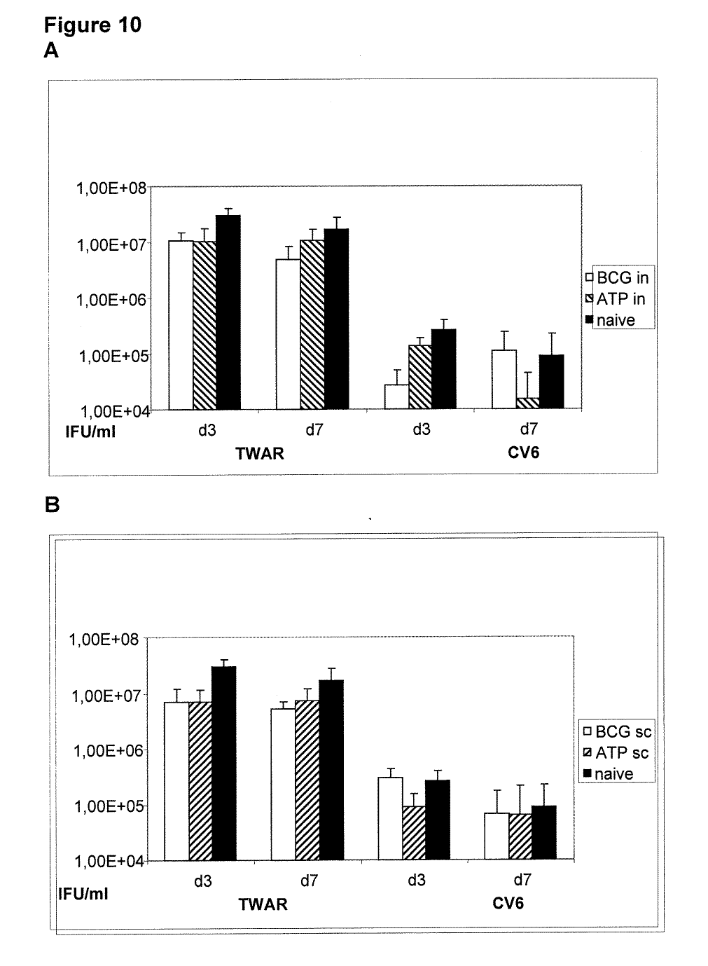

The infective titre of C. pneumoniae in the lungs of mice was determined by culturing lung homogenates on Hep2 cells (FIG. 10). The titre of TWAR in the lungs of naïve mice was significantly higher than in the lungs of BCG-immunised mice at the day 3 time point (BCG i.n. p<0.001; BCG-ATP i.n. p=0.001; BCG s.c. p<0.001; BCG-ATP s.c. p<0.001). At the day 7 time point the titre of TWAR strain was significantly higher in non-immunised mice than in BCG i.n. immunised mice (p=0.043) and higher than in BCG s.c. immunised mice (p=0.03). However, the difference was not significant when the C. pneumoniae titre of non-immunised mice was compared to the titre in the lungs of BCG-ATP i.n. (p=0.345) and BCG-ATP s.c. (p=0.081) immunised mice. The differences between BCG-immunised and non-immunised mice challenged with CV-6 strain of C. pneumoniae has not been evaluated statistically.

C. pneumoniae-Specific Serum IgG

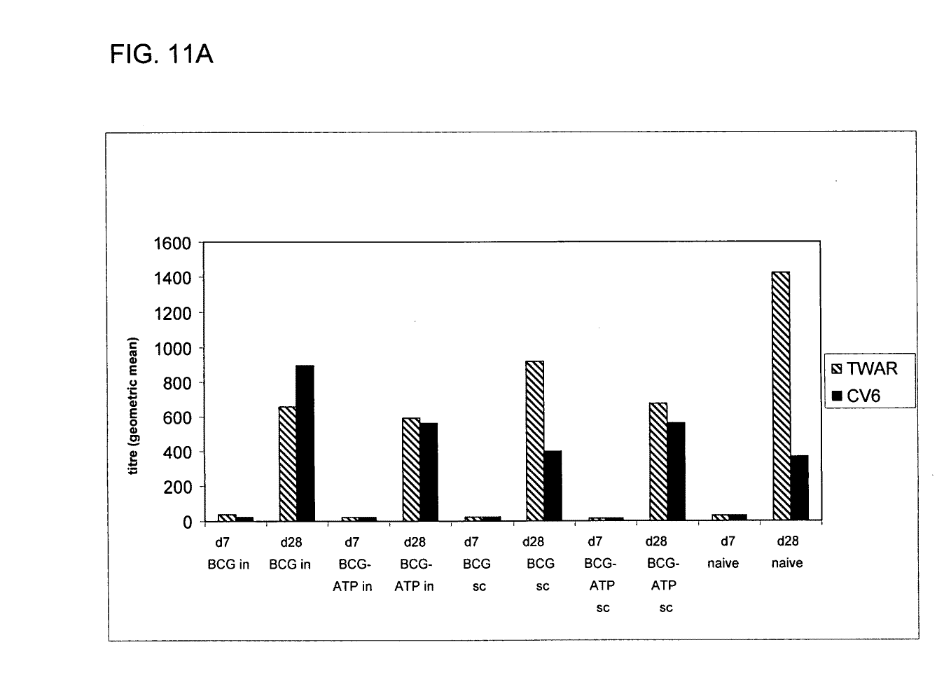

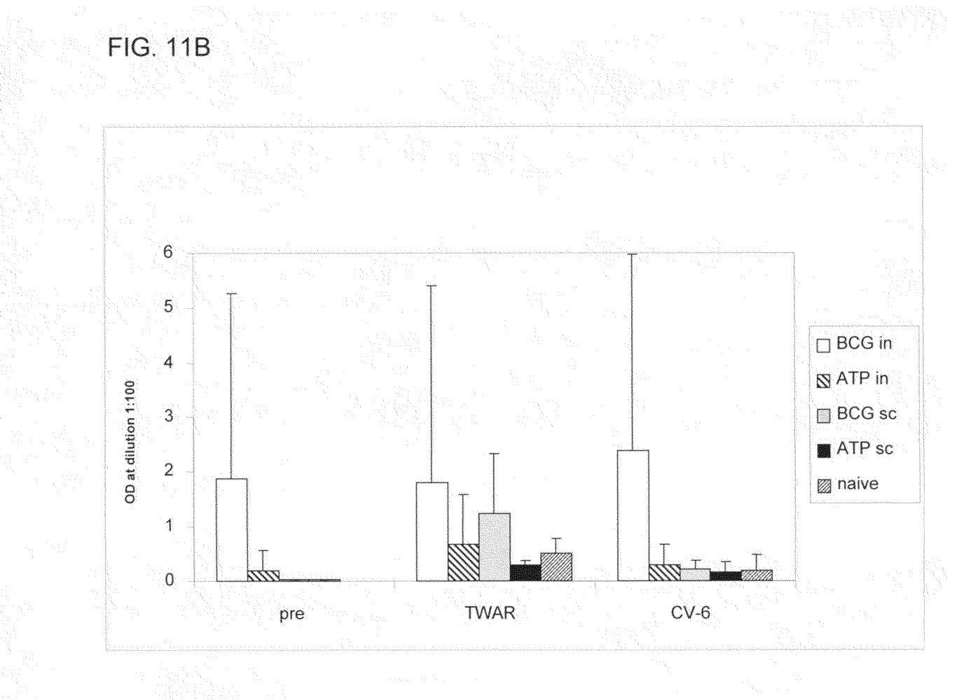

C. pneumoniae-specific serum IgG levels of BCG-immunised and C. pneumoniae challenged mice were compared to that of non-immunised C. pneumoniae-infected mice (FIG. 11). The geometric mean of IgG titre was higher in non-immunised TWAR-infected mice than in BCG-immunised mice. CV-6 infected non-immunised mice produced C. pneumoniae-specific IgG antibodies in lower titres than BCG-immunised mice.

### Chlamydial HSP60-Specific IgG Antibodies

Chlamydial HSP60-specific IgG antibodies were detected in serum samples collected at the day 28 time-point after BCG immunisation or C. pneumoniae challenge by using ELISA plates coated with cHSP-60 (FIG. 11B). Mice inoculated twice with parental BCG intranasally produced antibodies reacting with cHSP60, fewer mice in rBCG-‘ATP’ immunised group produced this antibody. The level of the antibody was equally high in mice immunised intranasally with parental BCG pre-challenge or after TWAR or CV-6 challenge. In other BCG-immunised groups after C. pneumoniae challenge the frequency and level of cHSP60-specific antibodies increased in TWAR-challenged mice, and moderately in CV-6 challenged mice.

C. pneumoniae-Specific IgA

At the day 28 time-point after C. pneumoniae challenge, mouse sera were tested for the presence of C. pneumoniae-specific IgA antibody. The frequency and level of IgA antibody was similar in non-immunised TWAR-infected mice and in mice immunised i.n. with parental BCG. In most of the non-immunised and BCG-immunised CV-6 infected mice the C. pneumoniae-specific serum IgA level remained below detectable level.

C. pneumoniae-Specific IgG in Lung

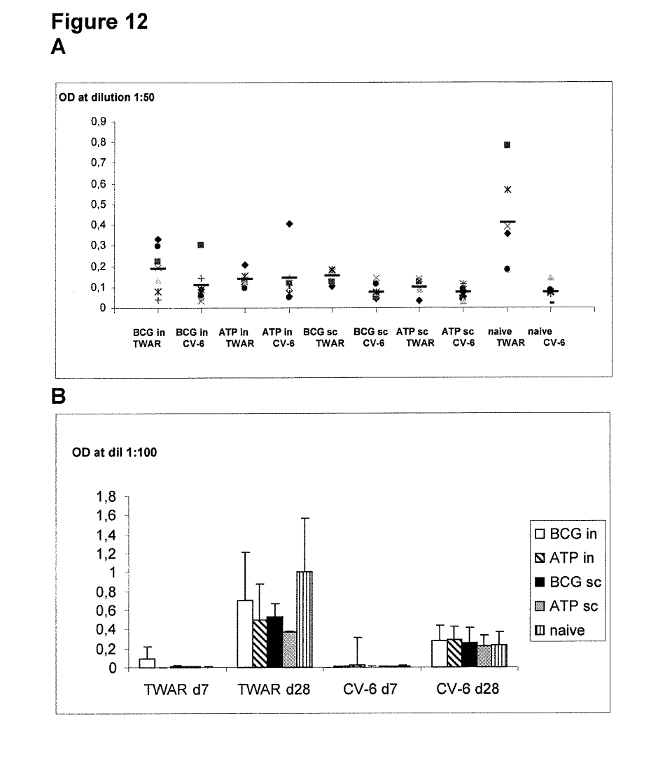

C. pneumoniae-specific IgG antibodies were also detectable in the lung homogenates of the mice at the day 28 time-point after C. pneumoniae challenge (FIG. 12A). The highest level was demonstrated in the non-immunised TWAR-infected mice.

C. pneumoniae-Specific IgA in Lung

Levels of the local C. pneumoniae-specific IgA antibodies in the lungs (FIG. 12B) did not increase in response to BCG-immunisation compared to that of non-immunised C. pneumoniae-challenged mice. The IgA antibodies were produced in larger quantities in TWAR-challenged than in CV-6 challenged mice.

### Antigen-Specific Spleen Cell Proliferation

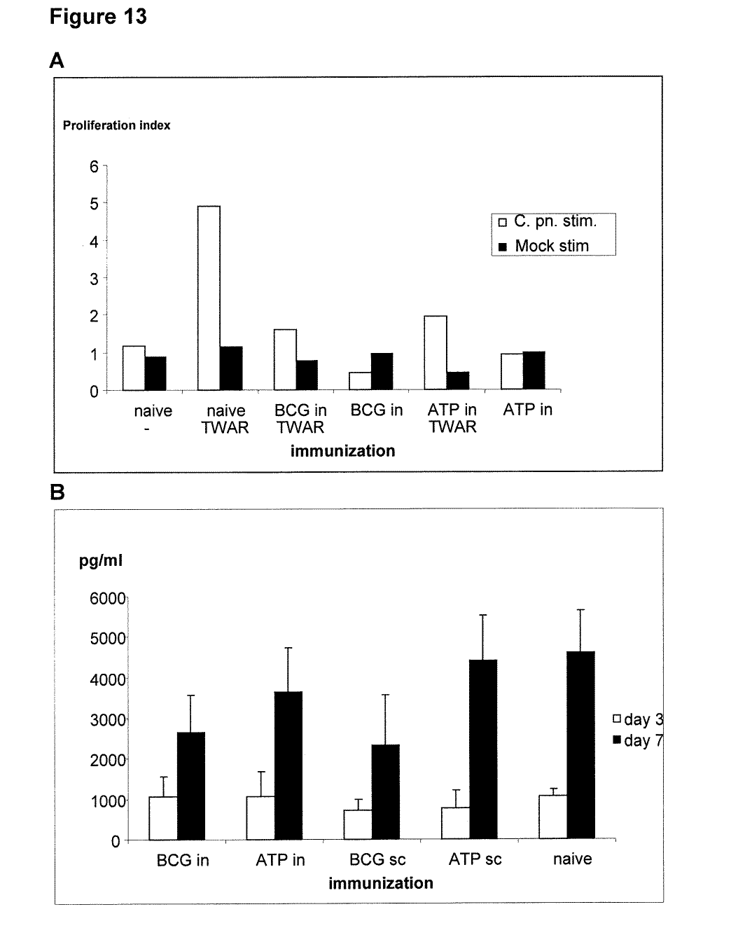

Spleen cells of BCG-immunised and non-immunised mice without C. pneumoniae challenge or after C. pneumoniae (TWAR) challenge were stimulated with formalin-fixed C. pneumoniae elementary bodies or with control antigen. The proliferation index was determined by using AlamarBlue reagent (FIG. 13A). The proliferation index was the highest in case of non-immunised TWAR-infected mice. In mice immunised with BCG or BCG-ATP and challenged with TWAR, the proliferation index exceeded that of naïve mice and non-challenged but immunised mice, however did not reach the value of proliferation index in non-immunised TWAR-infected mice.

### IFN-γ Production

To investigate the local cellular immune response, lung homogenates of BCG and BCG-ATP immunised and non-immunised mice challenged with C. pneumoniae were tested for IFN-γ production (FIG. 13B). After TWAR-challenge the IFN-γ level was highest on day 7. IFN-γ was below detectable level on day 28. In BCG-ATP immunised mice the lung IFN-γ level was close to that of non-immunised mice however, parental BCG-immunised mice produced smaller amount of IFN-γ than non-immunised mice.

Infection with CV-6 strain induced low level of IFN-γ in some mice, and in majority of mice the IFN-γ was not detectable.

### IL-6 Levels in Lung

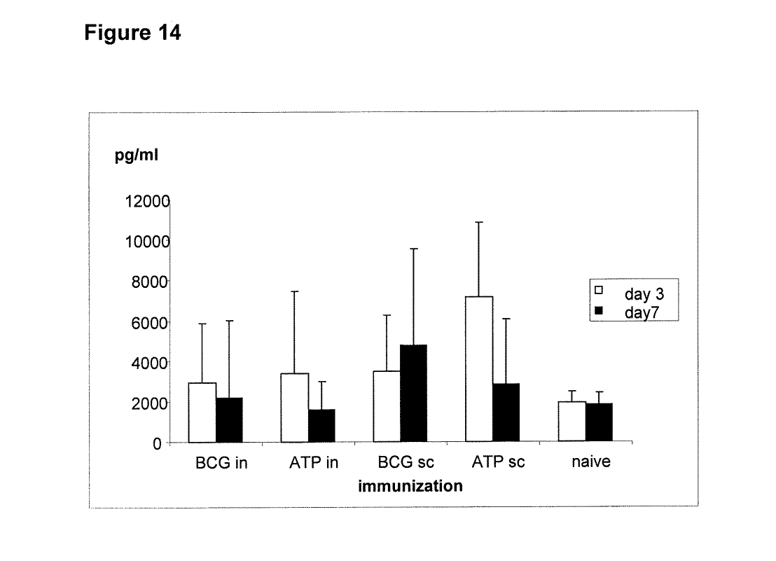

IL-6, the cytokine produced during infection with an important role in inflammation was detected in the lung homogenates of the naïve, BCG-immunised and C. pneumoniae-challenged mice. In case of TWAR-challenge BCG immunised mice produced slightly higher level of IL-6 in their lungs than the non-immunised mice, the difference was more pronounced at day 3 time-point (FIG. 14).

In CV-6 infected mice IL-6 production showed large individual differences within BCG-immunisation groups, it was generally at low level, especially at day 7.

### Example 7

### Immunization with LcrE Protein

LcrE of C. pneumoniae TWAR (also mentioned as Chlamydial outer protein N-CopN) was amplified by PCR and cloned into pET vector carrying His and FLAG tags. The protein was over-expressed in E. coli HB101 and purified by using HIS-select cartridge. LcrE protein was also used for immunization to test the protective effect of the expressed protein. In our experiments we compared the immunogenicity of LcrE protein in combination with Alum the most widely used adjuvant in humans with that of LcrE mixed with Freund's adjuvant which is potent but too reactogenic to use in humans (Guy, 2007 Nat Rev Microbiol. July; 5(7):505-17. Review). BALB/c mice were immunized subcutaneously with the purified LcrE protein at a dose of 20 μg mixed with Alum adjuvant or Freund's adjuvants (1^{st} inoculation with complete and 2^{nd} and 3^{rd} inoculations with incomplete Freund's adjuvant) 3 times at 3-week intervals. Two weeks after the last immunization the immunized and non-immunized mice were challenged with 4×10⁵ IFU C. pneumoniae intranasally. Mice were sacrificed 7 days after infection. Lungs were excised and tested for viable C. pneumoniae by culture. Sera were tested for LcrE-specific IgG, IgG1, IgG2a, IgA by ELISA. IgA and cytokines (IL-4, IL-6, IL-10 and IFN-γ) were measured in the lungs. Cellular immune response was assessed by detection of cytokines produced by spleen cells after in vitro re-stimulation with LcrE protein or C. pneumoniae. Lymphocyte proliferation was tested by MTT test. The number of IFN-γ producing spleen cells upon LcrE or C. pneumoniae stimulation was determined by ELISPOT assay.

### Results

Protection Against C. pneumoniae Infection

Protection against C. pneumoniae infection was measured by culture of C. pneumoniae from the lungs—

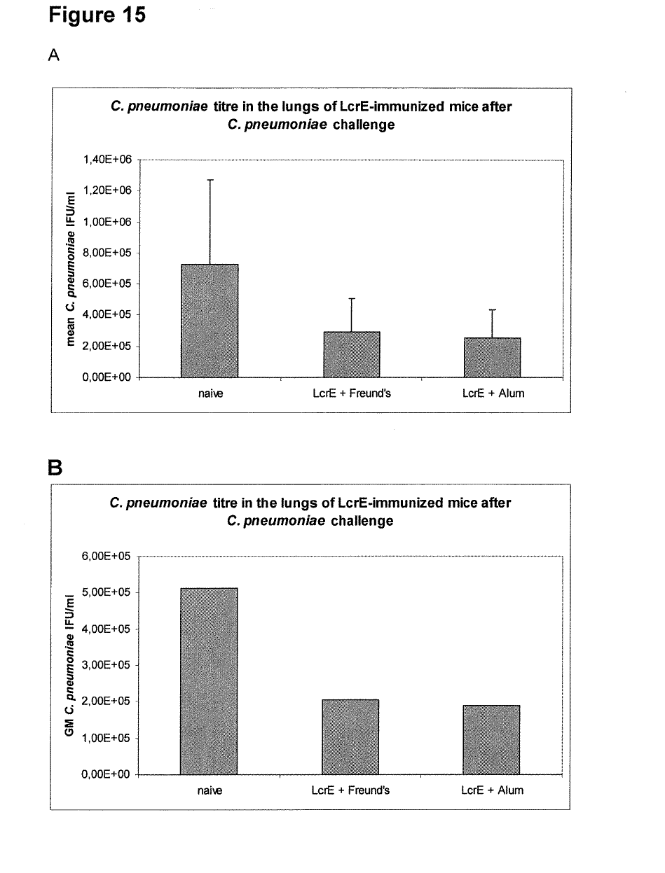

A significant reduction in the number of C. pneumoniae cultured from the lungs after C. pneumoniae challenge was detected. The reduction in the mean titre of C. pneumoniae in the lungs compared to non-immunized mice was 60% (60% when calculated as geometric mean) when Freund's adjuvant was used and 65% (63% when calculated as geometric mean) when Alum adjuvant was used (FIG. 15 A, B).

### LcrE-Specific Antibodies in the Sera of LcrE-Immunized Mice

High-titre LcrE-specific IgG was detected in the mouse sera at the time of C. pneumoniae challenge, no significant difference was observed between the antibody levels induced by the different adjuvants.

TABLE 2
LcrE-specific IgG antibody levels
induced by the different adjuvants

| Immunization | LcrE-specific IgG titre GM |
|---|---|
| LcrE + Freund's | 172216 |
| LcrE + Alum | 164540 |

### IgG1 and IgG2a Sera Levels

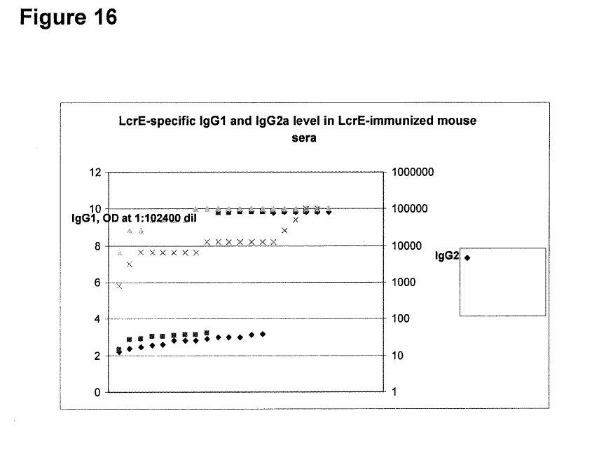

Higher IgG2a titre was induced by using Freund's adjuvant and higher level of IgG1 was present after Alum-immunization (FIG. 16). Higher relative IgG2a level suggests Th1 type immune response, high IgG1 level refers to Th2 type immune response. Irrespective of the applied adjuvant high IgG1 level was detected in the sera, however in Freund's adjuvant-immunized mice the IgG2a titres were higher.

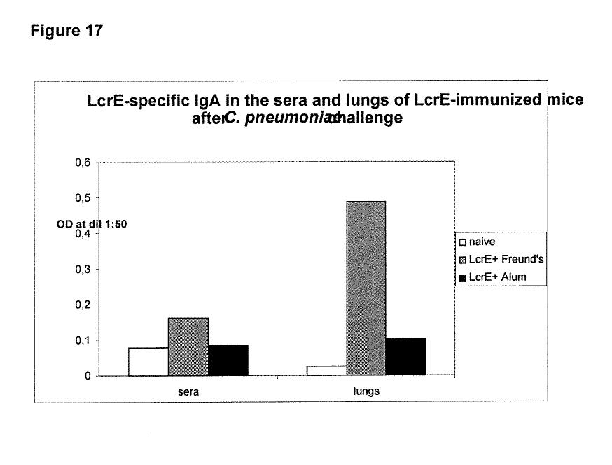

LcrE-specific IgA level was measured in the sera and in the lungs (FIG. 17). Freund's adjuvant was more effective in inducing LcrE-specific IgA at both sites.

### Local Cytokine Response

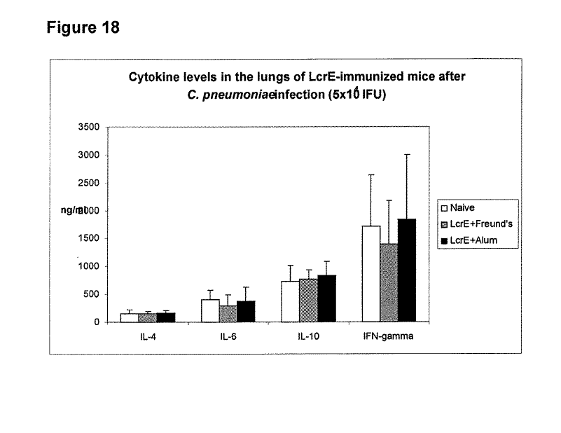

The local cytokine response was analyzed by detecting IL-4, IL-6, IL-10 and IFN-γ in the supernatant of lung homogenates (FIG. 18). IL-4 was present at marginal level only in the lungs of LcrE+Alum immunized mice. IL-10 was also slightly increased in LcrE+Alum immunized mouse lungs. Detection of these cytokines suggests Th2 type immune response. The IL-6 and IFN-γ content of the lung was diminished in the LcrE+Freund's immunized mice which might suggest a lower level of inflammation in these mice.

### Cellular Immune Response

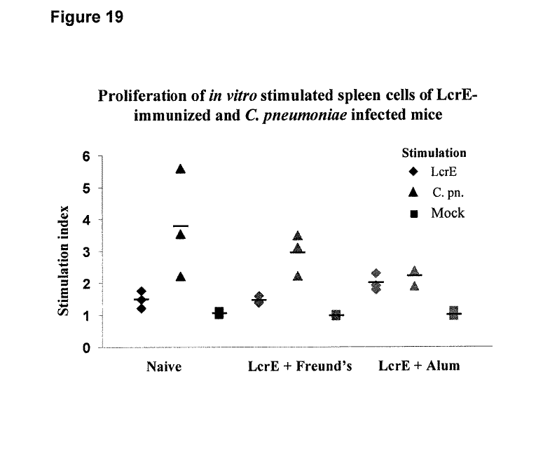

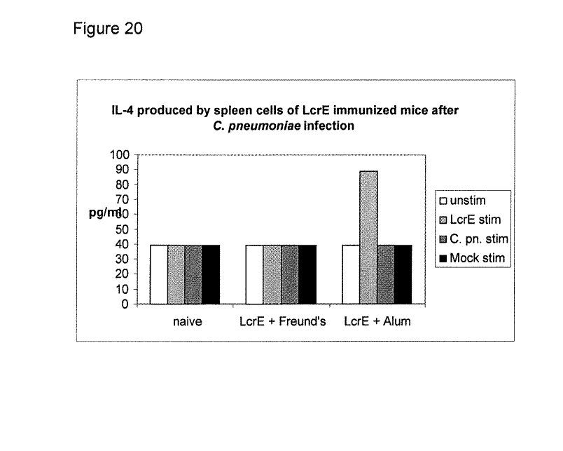

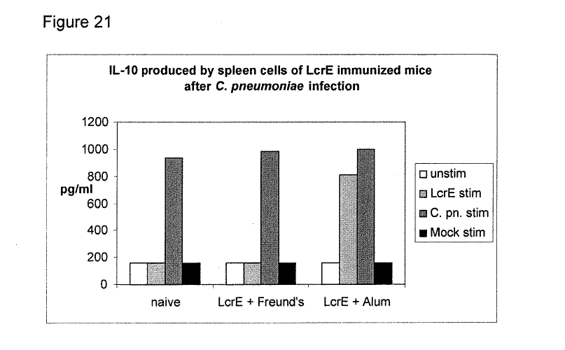

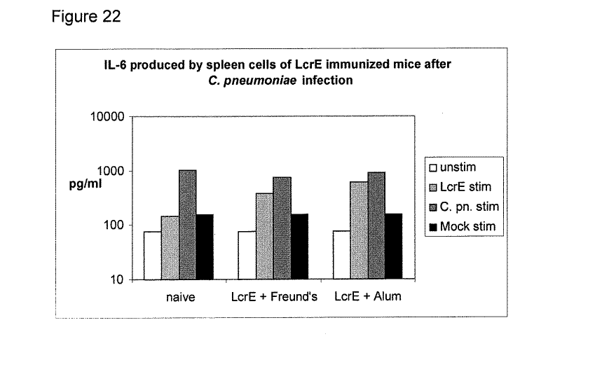

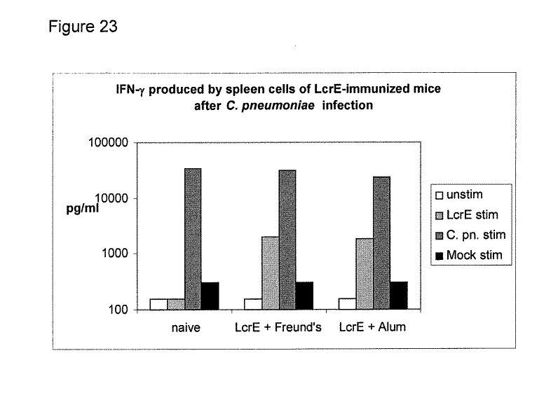

Cellular immune response was assayed by in vitro stimulation of the spleen cells of the LcrE immunized and naïve mice 7 days after C. pneumoniae infection (FIG. 19). Stimulation with LcrE protein induced proliferation of non-immunized (naïve) mice also, and LcrE+Alum immunized mice displayed increased proliferative response. Stimulation with C. pneumoniae antigen resulted in a lower proliferation index in LcrE+Alum immunized mice compared to non-immunized and LcrE+Freund's immunized mice. Supernatant of in vitro stimulated spleen cells was tested for production of cytokines (IL-4, FIG. 20; IL-10, FIG. 21; IL-6, FIG. 22; IFN-γ, FIG. 23) by ELISA assays.

Enumeration of C. pneumoniae Antigen-Specific IFN-γ Secreting Cells.

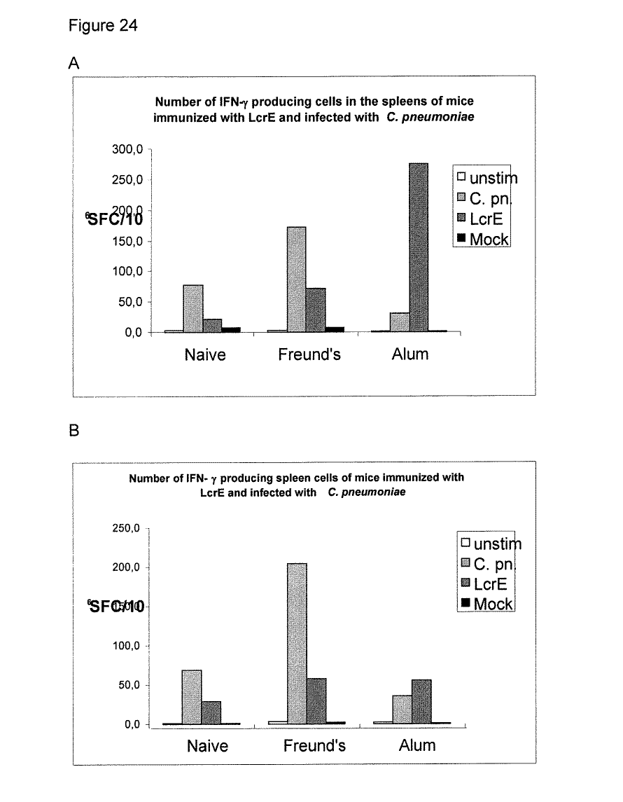

ELISPOT assay was set up to enumerate the LcrE and whole C. pneumoniae antigen-specific IFN-γ secreting cells, the results of which are illustrated in FIGS. 24a and b.

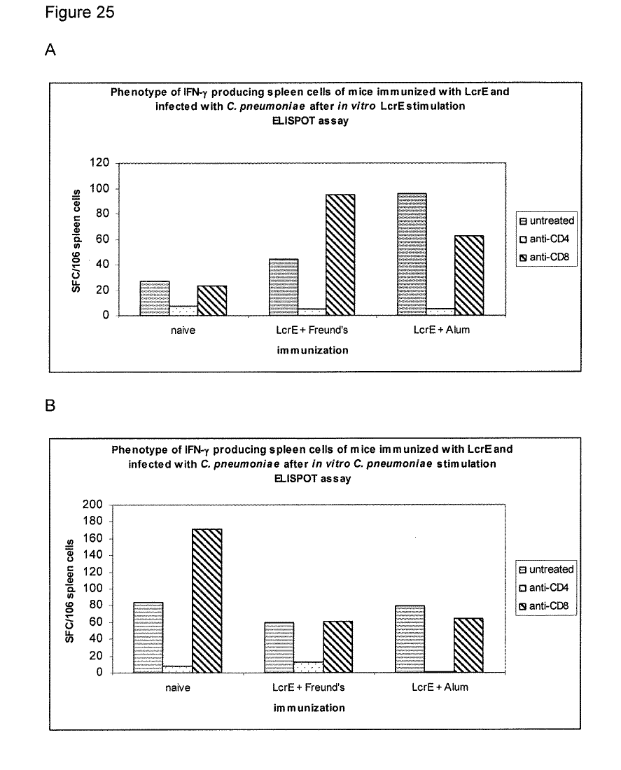

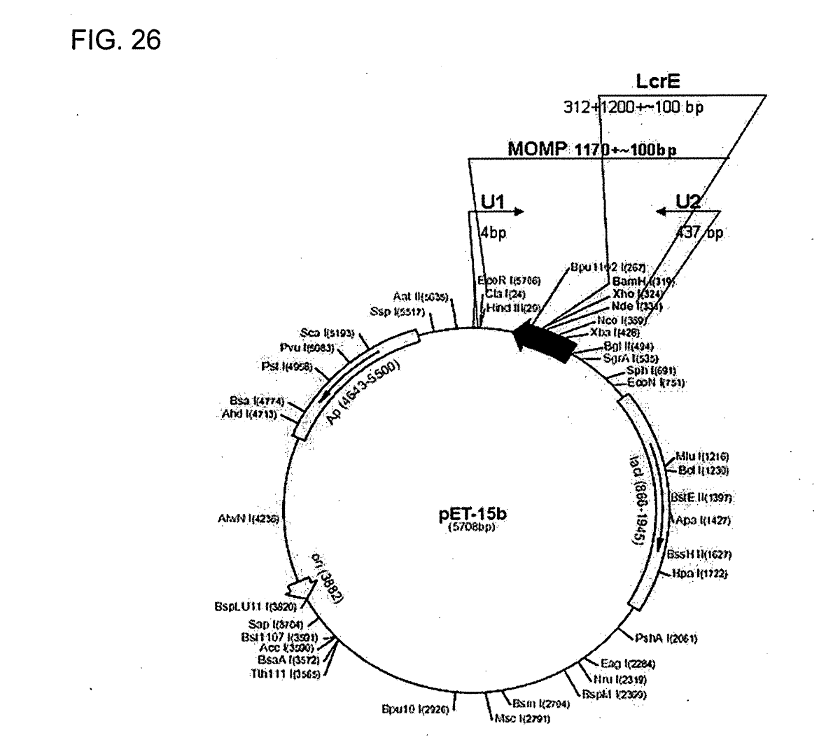

ELISPOT assay was performed to define the phenotype of spleen cells producing IFN-γ in mice immunized with LcrE protein and infected subsequently with C. pneumoniae (FIG. 25 A, B). Spleen cell suspensions were depleted of CD4⁺ and CD8⁺ cells, respectively by using micro-beads coated with the respective antibody and applying the magnetic cell sorting (MACS) system of Miltenyi Biotec Inc. The outcome of the procedure was tested by FACS analysis after direct staining of the depleted cells by α-CD4-TC and α-CD8-rPE antibodies. Significant decrease in the number of LcrE-stimulated and also C. pneumoniae-stimulated IFN-γ producing cells was observed after removal of CD4+ cells but not after depletion of CD8⁺ cells.

### Example 8

### Construction of LcrE and MOMP Vectors

LcrE (chlamydial outer protein N-CopN, gene: pCB0334) and MOMP (major outer membrane protein, gene: CpB0722) of C. pneumoniae have been cloned into pET E. coli expression plasmid. Primers were designed that are universal for amplification from pET based constructs in order to clone the chlamydial genes into an E. coli-mycobacterium shuttle vector, pJH154 (Yu. et al., 2006 Clin Vaccine Immunol. 11:1204-11). pJH plasmids are kanamycin resistant and were designed to express foreign proteins in different locations under regulation of the M. tuberculosis α-antigen promoter. The inserts in pJH154 plasmid contain the His and FLAG tags. BCG and M. smegmatis were transformed with this constructs.

1. A recombinant Mycobacterium bovis which expresses at least one Chlamydophila pneumoniae antigen.

2. A recombinant Mycobacterium bovis according to claim 1, wherein said antigen comprises at least an antigenically distinct part of one or more of: CopN, CPAF, ADP/ATP Translocase I, SmpB, pmpD, MOMP, LcrH1, LcrH2, OMP2 and HSP60.

3. A recombinant Mycobacterium bovis according to claim 1, wherein said antigen comprises at least an antigenically distinct part of ADP/ATP translocase 1.

4. A recombinant Mycobacterium bovis according to claim 1, wherein the Mycobacterium bovis expresses an antigenically distinct part of a polypeptide selected from the group consisting of:
i) a polypeptide having an amino acid sequence at least 70% identical to SEQ ID NO:1;
ii) a polypeptide having an amino acid sequence at least 70% identical to SEQ ID NO:2;
iii) a polypeptide having an amino acid sequence at least 70% identical to SEQ ID NO:3;
iv) a polypeptide having an amino acid sequence at least 70% identical to SEQ ID NO:4;
v) a polypeptide having an amino acid sequence at least 70% identical to SEQ ID NO:5;
vi) a polypeptide having an amino acid sequence at least 70% identical to SEQ ID NO:6;
vii) a polypeptide having an amino acid sequence at least 70% identical to SEQ ID NO:11;
viii) a polypeptide having an amino acid sequence at least 70% identical to SEQ ID NO:12;
ix) a polypeptide having an amino acid sequence at least 70% identical to SEQ ID NO:13; and
x) a polypeptide having an amino acid sequence at least 70% identical to SEQ ID NO:14.

5. A recombinant Mycobacterium bovis according to claim 4, wherein the Mycobacterium bovis expresses an antigenically distinct part of a polypeptide selected from the group consisting of:
i) a polypeptide having an amino acid sequence of SEQ ID NO:1;
ii) a polypeptide having an amino acid sequence of SEQ ID NO:2;
iii) a polypeptide having an amino acid sequence of SEQ ID NO:3;
iv) a polypeptide having an amino acid sequence of SEQ ID NO:4;
v) a polypeptide having an amino acid sequence of SEQ ID NO:5;
vi) a polypeptide having an amino acid sequence of SEQ ID NO:6;
vii) a polypeptide having an amino acid sequence of SEQ ID NO:11;
viii) a polypeptide having an amino acid sequence of SEQ ID NO:12;
ix) a polypeptide having an amino acid sequence of SEQ ID NO:13; and
x) a polypeptide having an amino acid sequence of SEQ ID NO:14.

6. A recombinant Mycobacterium bovis according to claim 1, wherein said antigen is secreted from said organism.

7. A recombinant Mycobacterium bovis according to claim 1, wherein said antigen is expressed as a membrane-bound form on the surface of said organism.

8. A recombinant Mycobacterium bovis according to claim 1, which is a BCG organism.

9. An Escherichia coli-Mycobacterium bovis shuttle vector comprising a nucleic acid encoding at least an antigenically distinct part of one of one or more Chlamydophila pneumoniae antigens selected from the group consisting of: CopN, CPAF, ADP/ATP Translocase I, SmpB, pmpD, MOMP, LcrH1, LcrH2, OMP2 and HSP60.

10. An Escherichia coli-Mycobacterium bovis shuttle vector according to claim 9, comprising one or more nucleic acid molecules selected from the group consisting of:
i) a nucleic acid molecule comprising a nucleotide sequence having at least 70% identity to a nucleotide sequence of SEQ ID NO:15;
ii) a nucleic acid molecule comprising a nucleotide sequence having at least 70% identity to a nucleotide sequence of SEQ ID NO:16;
iii) a nucleic acid molecule comprising a nucleotide sequence having at least 70% identity to a nucleotide sequence of SEQ ID NO:17;
iv) a nucleic acid molecule comprising a nucleotide sequence having at least 70% identity to a nucleotide sequence of SEQ ID NO:18;
v) a nucleic acid molecule comprising a nucleotide sequence having at least 70% identity to a nucleotide sequence of SEQ ID NO:19;
vi) a nucleic acid molecule comprising a nucleotide sequence having at least 70% identity to a nucleotide sequence of SEQ ID NO:20;
vii) a nucleic acid molecule comprising a nucleotide sequence having at least 70% identity to a nucleotide sequence of SEQ ID NO:21;
viii) a nucleic acid molecule comprising a nucleotide sequence having at least 70% identity to a nucleotide sequence of SEQ ID NO:22;
ix) a nucleic acid molecule comprising a nucleotide sequence having at least 70% identity to a nucleotide sequence of SEQ ID NO:23; and
x) a nucleic acid molecule comprising a nucleotide sequence having at least 70% identity to a nucleotide sequence of SEQ ID NO:24.

11. An Escherichia coli-Mycobacterium bovis shuttle vector according to claim 9, wherein said nucleic acid molecule is selected from the group consisting of:
i) a nucleic acid molecule comprising a nucleotide sequence of SEQ ID NO:15;
ii) a nucleic acid molecule comprising a nucleotide of SEQ ID NO:16;
iii) a nucleic acid molecule comprising a nucleotide sequence of SEQ ID NO: 17;
iv) a nucleic acid molecule comprising a nucleotide sequence of SEQ ID NO:18;
v) a nucleic acid molecule comprising a nucleotide sequence of SEQ ID NO:19;
vi) a nucleic acid molecule comprising a nucleotide sequence of SEQ ID NO:20;
vii) a nucleic acid molecule comprising a nucleotide of SEQ ID NO:21;
viii) a nucleic acid molecule comprising a nucleotide sequence of SEQ ID NO:22;
ix) a nucleic acid molecule comprising a nucleotide sequence of SEQ ID NO:23; and
x) a nucleic acid molecule comprising a nucleotide sequence of SEQ ID NO:24.

12. An Escherichia coli-Mycobacterium bovis shuttle vector according to claim 9, comprising a nucleic acid molecule which encodes a polypeptide selected from the group consisting of:
i) a polypeptide having an amino acid sequence at least 70% identical to SEQ ID NO:1;
ii) a polypeptide having an amino acid sequence at least 70% identical to SEQ ID NO:2;
iii) a polypeptide having an amino acid sequence at least 70% identical to SEQ ID NO:3;
iv) a polypeptide having an amino acid sequence at least 70% identical to SEQ ID NO:4;
v) a polypeptide having an amino acid sequence at least 70% identical to SEQ ID NO:5;
vi) a polypeptide having an amino acid sequence at least 70% identical to SEQ ID NO:6;
vii) a polypeptide having an amino acid sequence at least 70% identical to SEQ ID NO:11;
viii) a polypeptide having an amino acid sequence at least 70% identical to SEQ ID NO:12;
ix) a polypeptide having an amino acid sequence at least 70% identical to SEQ ID NO:13; and
x) a polypeptide having an amino acid sequence at least 70% identical to SEQ ID NO:14.

13. An Escherichia coli-Mycobacterium bovis shuttle vector according to claim 9, wherein said nucleic acid molecule encodes a polypeptide selected from the group consisting of:
i) a polypeptide having an amino acid sequence of SEQ ID NO:1;
ii) a polypeptide having an amino acid sequence of SEQ ID NO:2;
iii) a polypeptide having an amino acid sequence of SEQ ID NO:3;
iv) a polypeptide having an amino acid sequence of SEQ ID NO:4;
v) a polypeptide having an amino acid sequence of SEQ ID NO:5;
vi) a polypeptide having an amino acid sequence of SEQ ID NO:6;
vii) a polypeptide having an amino acid sequence of SEQ ID NO:11;
viii) a polypeptide having an amino acid sequence of SEQ ID NO:12;
ix) a polypeptide having an amino acid sequence of SEQ ID NO:13; and
x) a polypeptide having an amino acid sequence of SEQ ID NO:14.

14. A Mycobacterium bovis cell transformed or transfected with an Escherichia coli-Mycobacterium bovis shuttle vector according to claim 9.

15. A vaccine comprising the recombinant Mycobacterium bovis a of claim 1, wherein optionally the vaccine further comprises a pharmaceutically acceptable adjuvant.

16. A vaccine comprising the Mycobacterium bovis cell according to claim 14, wherein optionally the vaccine further comprises a pharmaceutically acceptable adjuvant.

17. A vaccine comprising the Escherichia coli-Mycobacterium bovis shuttle vector of claim 9, wherein optionally the vaccine further comprises a pharmaceutically acceptable adjuvant.

18. A method of immunizing a subject against a pathogenic microbe of the genus Chlamydophila or treating, preventing or reducing a Chlamydophila pneumoniae infection comprising administering a therapeutically effective amount of a vaccine according to claim 15 to a subject.

19. A method of immunizing a subject against a pathogenic microbe of the genus Chlamydophila or treating, preventing or reducing a Chlamydophila pneumoniae infection comprising administering a therapeutically effective amount of a vaccine according to claim 16 to a subject.

20. A method of immunizing a subject against a pathogenic microbe of the genus Chlamydophila or treating, preventing or reducing a Chlamydophila pneumoniae infection comprising administering a therapeutically effective amount of a vaccine according to claim 17 to a subject.

21. The method according to claim 18, wherein said pathogenic microbe is Chlamydophila pneumoniae.

22. The method according to claim 19, wherein said pathogenic microbe is Chlamydophila pneumoniae.

23. The method according to claim 20, wherein said pathogenic microbe is Chlamydophila pneumoniae.

24. A method of preventing or reducing atherosclerosis comprising administering a therapeutically effective amount of the vaccine of claim 15 to a subject.

25. A method of preventing or reducing atherosclerosis comprising administering a therapeutically effective amount of the vaccine of claim 16 to a subject.

26. A method of preventing or reducing atherosclerosis comprising administering a therapeutically effective amount of the vaccine of claim 17 to a subject.

27. A method of preventing or reducing atheroma comprising administering a therapeutically effective amount of the vaccine of claim 15 to a subject.

28. A method of preventing or reducing atheroma comprising administering a therapeutically effective amount of the vaccine of claim 16 to a subject.

29. A method of preventing or reducing atheroma comprising administering a therapeutically effective amount of the vaccine of claim 17 to a subject.

30. A method of preventing or retarding atherosclerotic cardiovascular disease comprising administering a therapeutically effective amount of the vaccine of claim 15 to a subject.

31. A method of preventing or retarding atherosclerotic cardiovascular disease comprising administering a therapeutically effective amount of the vaccine of claim 16 to a subject.

32. A method of preventing or retarding atherosclerotic cardiovascular disease comprising administering a therapeutically effective amount of the vaccine of claim 17 to a subject.
